SEQUENCE LISTING (2 sequences)

SEQ ID NO: 1 - protein, 120 aa, Artificial Sequence
  Synthetic Construct
QVQLVQSGAEVKKPGASVKVSCKASGLTIEDYYMHWVRQAPGQGLEWMGWIDPENGDTEYGPKFQGRVTMTRDTSINTAYMELSRLRSDDTAVYYCAVHNAHYGTWFAYWGQGTLVTVSS

SEQ ID NO: 2 - protein, 113 aa, Artificial Sequence
  Synthetic Construct
DVVMTQSPLSLPVTLGQPASISCRSSQSLLHSSGNTYLEWYQQRPGQSPRPLIYKISTRFSGVPDRFSGSGSGTDFTLKISRVEAEDVGVYYCFQGSHVPYTFGGGTKVEIKR

(19) United States
(12) Patent Application Publication
(10) Pub. No.: US 2020/0283540 A1
(43) Pub. Date: Sep. 10, 2020

(54) HUMANIZED ANTI-LIV1 ANTIBODIES FOR THE TREATMENT OF BREAST CANCER

(71) Applicant: Seattle Genetics, Inc., Bothell, WA (US)

(72) Inventors: Dana KENNEDY, Kirkland, WA (US); Ana KOSTIC, Seattle, WA (US); Elizabeth CORWIN, Seattle, WA (US); Jonathan DRACHMAN, Seattle, WA (US); Peter HAUGHNEY, Seattle, WA (US); Baiteng ZHAO, Woodinville, WA (US); Phillip GARFIN, Bothell, WA (US); Corinna PALANCA-WESSELS, Bothell, WA (US); Oyewale O. ABIDOYE, Bellevue, WA (US)

(21) Appl. No.: 16/768,020

(22) PCT Filed: Nov. 30, 2018
(86) PCT No.: PCT/US2018/063425

§ 371 Date: May 28, 2020

Related U.S. Application Data
(60) Provisional application No. 62/593,660, filed on Dec. 1, 2017.

(51) Int. Cl. C07K 16/30; A61K 38/19; A61K 39/395; A61K 47/68

(52) U.S. Cl. CPC C07K 16/3015; A61K 38/193; A61K 2039/505; A61K 47/6803; A61K 47/6855; A61K 39/3955

(57) ABSTRACT

Methods for using anti-LIV1 antibodies, including dmg conjugated anti-LIV1 antibodies, to inhibit proliferation of a LIV-1-expressing cell, as well as for the treatment of one or more diseases or disorders associated with LIV-1-expressing cells (e.g., a LIV-1-associated breast cancer), are provided.

### CROSS-REFERENCE TO RELATED APPLICATION

This application claims priority to U.S. Provisional application No. 62/593,660 filed on Dec. 1, 2017, the content of which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761682001440SEQLIST.TXT, date recorded: Nov. 30, 2018, size: 3 KB).

### FIELD OF THE INVENTION

The present invention relates to the field of antibody-based breast cancer therapeutics. In particular, the present invention relates to the use of humanized anti-LIV1 antibodies and antigen-binding fragments or conjugates thereof (e.g., LIV1-antibody-drug conjugates (LIV1-ADCs)) for the treatment of LIV-1-expressing cancers, such as, e.g., breast cancer (e.g., locally advanced or metastatic breast cancer).

### BACKGROUND

Breast cancers are classified on the basis of three protein expression markers: estrogen receptor (ER), progesterone receptor (PgR), and the overexpression of the growth factor receptor HER2/neu. Hormonal therapies, including tamoxifen and aromatase inhibitors, can be effective in treating tumors that express the hormone receptors ER and PgR. HER2-directed therapies are useful for tumors that express HER2/neu; these tumors are the only class of breast cancer that is currently eligible for immunotherapy. For these patients, unconjugated antibodies, such as Herceptin or Perj eta, are generally used in combination with chemotherapy.

The treatment options for triple-negative breast tumors, those that do not express ER, PgR, or HER2/neu, are restricted to chemotherapy, radiation and surgery. In addition, there are limited effective treatment options available to patients with advanced-stage disease with relatively poor survival rates of stage III patients (52%) and significantly worse for stage IV patients (15%).

There is clearly a significant need for effective treatments for breast cancer, particularly late-stage breast cancer.

LIV-1 (SLC39A6) is a member of the solute carrier family, a multi-span transmembrane protein with putative zinc transporter and metalloproteinase activity. LIV-1 was first identified as an estrogen-induced gene in the breast cancer cell line ZR-75-1. LIV-1 is expressed in most subtypes of metastatic breast cancer.

### SUMMARY

The present disclosure is based on the surprising discovery that incurable, unresectable, locally advanced or metastatic breast cancer can be treated with the anti-LIV1 antibodies and antigen-binding fragments thereof described herein.

In one aspect, a method of treating a subject having or at risk of having a LIV-1-associated cancer, comprising administering to the subject a therapeutically effective dose of an antibody or an antigen-binding fragment thereof that specifically binds human LIV-1, wherein the dose administered is less than about 200 mg of the antibody or antigen-binding fragment thereof per treatment cycle, and wherein the antibody or antigen-binding fragment thereof comprises a heavy chain variable region (HCVR) having at least 95% identity to SEQ ID NO: 1, and a light chain variable region (LCVR) having at least 95% identity to SEQ ID NO: 2, is provided.

In another aspect, a method of treating a subject having or at risk of having a LIV-1-associated cancer, comprising administering to the subject a therapeutically effective dose of an antibody or an antigen-binding fragment thereof that specifically binds human LIV-1, wherein the dose administered is less than or equal to about 250 mg of the antibody or antigen-binding fragment thereof per treatment cycle, wherein the antibody or antigen-binding fragment thereof comprises a heavy chain variable region (HCVR) having at least 95% identity to SEQ ID NO: 1, and a light chain variable region (LCVR) having at least 95% identity to SEQ ID NO: 2, and wherein if the dose administered is greater than or equal to about 200 mg of the antibody or antigen-binding fragment thereof per treatment cycle, the method further comprises administering granulocyte colony stimulating factor (GCSF) to the subject is provided. In certain exemplary embodiments, if administered, the GCSF is administered prophylactically.

In another aspect, a method of treating a subject having or at risk of having a LIV-1-associated cancer, comprising administering to the subject granulocyte colony stimulating factor (GCSF), administering to the subject a therapeutically effective dose of an antibody or an antigen-binding fragment thereof that specifically binds human LIV-1, wherein the dose administered is greater than or equal to about 200 mg and less than or equal to about 250 mg of the antibody or antigen-binding fragment thereof per treatment cycle, wherein the antibody or antigen-binding fragment thereof comprises a heavy chain variable region (HCVR) having at least 95% identity to SEQ ID NO: 1, and a light chain variable region (LCVR) having at least 95% identity to SEQ ID NO: 2 is provided. In certain exemplary embodiments, the GCSF is administered prophylactically.

In certain exemplary embodiments, the LIV-1-associated cancer is a breast cancer, a triple negative breast cancer, a metastatic breast cancer, a triple-negative, metastatic breast cancer or a hormone receptor-positive, metastatic breast cancer.

In certain exemplary embodiments, the treatment cycle is about every three weeks (Q3W).

In certain exemplary embodiments, the dose is about 2.5 mg/kg of body weight of the subject.

In certain exemplary embodiments, the antibody or antigen-binding fragment thereof is conjugated to monomethyl auristatin E (MMAE):

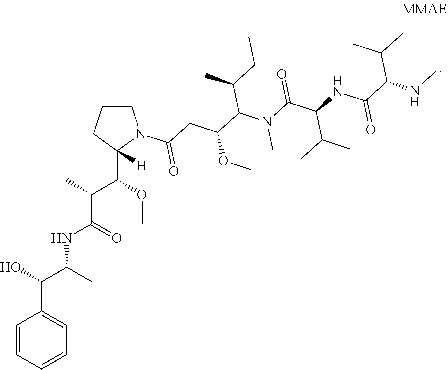 [SMILES: CC[C@H](C)C([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

In certain exemplary embodiments, the antibody or antigen-binding fragment thereof is conjugated to valine-citrulline-monomethyl auristatin E (vcMMAE):

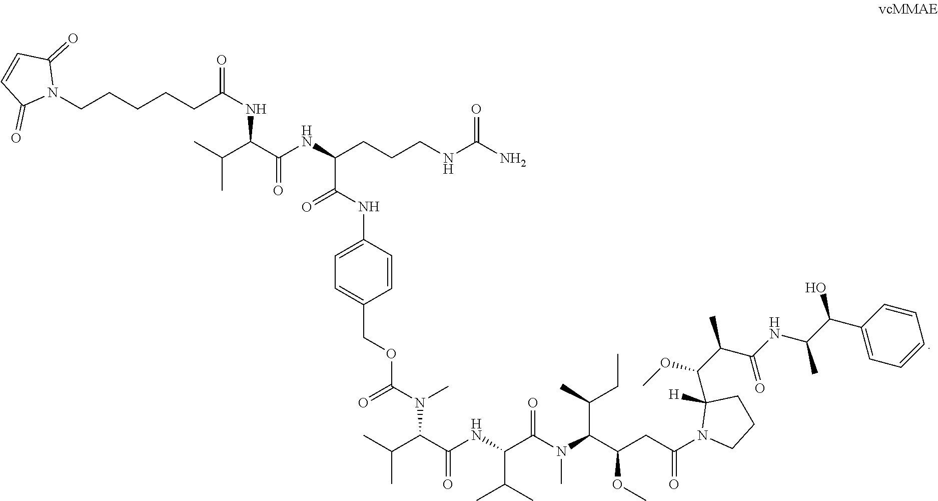 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

In certain exemplary embodiments, a vcMMAE to antibody or antigen-binding fragment thereof ratio is from about 1 to about 8 or about 4.

In certain exemplary embodiments, the HCVR has at least 97% sequence identity to SEQ ID NO: 1 and the LCVR has at least 97% sequence identity to SEQ ID NO: 2.

In certain exemplary embodiments, the HCVR has at least 99% sequence identity to SEQ ID NO: 1 and the LCVR has at least 99% sequence identity to SEQ ID NO: 2.

In certain exemplary embodiments, the subject is a human.

In another aspect, a method of treating a subject having or at risk of having a LIV-1-associated cancer, comprising administering to the subject a therapeutically effective dose of an antibody or an antigen-binding fragment thereof that specifically binds human LIV-1, wherein the dose administered is less than about 200 mg of the antibody or antigen-binding fragment thereof per treatment cycle, wherein the antibody or antigen-binding fragment thereof comprises an HCVR having at least 95% identity to SEQ ID NO: 1, and an LCVR having at least 95% identity to SEQ ID NO: 2, and wherein the antibody or antigen-binding fragment thereof is conjugated to vcMMAE:

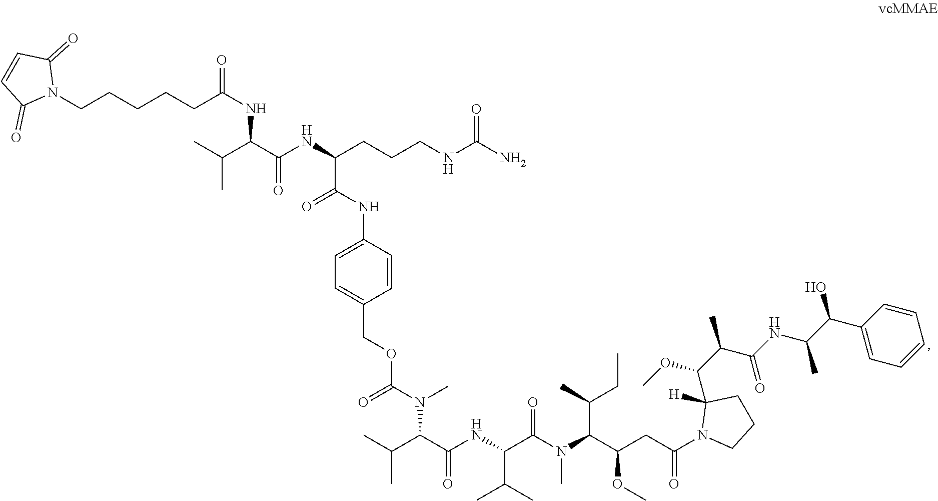 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

is provided.

In another aspect, a method of treating a subject having or at risk of having a LIV-1-associated cancer, comprising administering to the subject a therapeutically effective dose of an antibody or an antigen-binding fragment thereof that specifically binds human LIV-1, wherein the dose administered is less than or equal to about 250 mg of the antibody or antigen-binding fragment thereof per treatment cycle, wherein the antibody or antigen-binding fragment thereof comprises an HCVR having at least 95% identity to SEQ ID NO: 1, and an LCVR having at least 95% identity to SEQ ID NO: 2, wherein the antibody or antigen-binding fragment thereof is conjugated to vcMMAE:

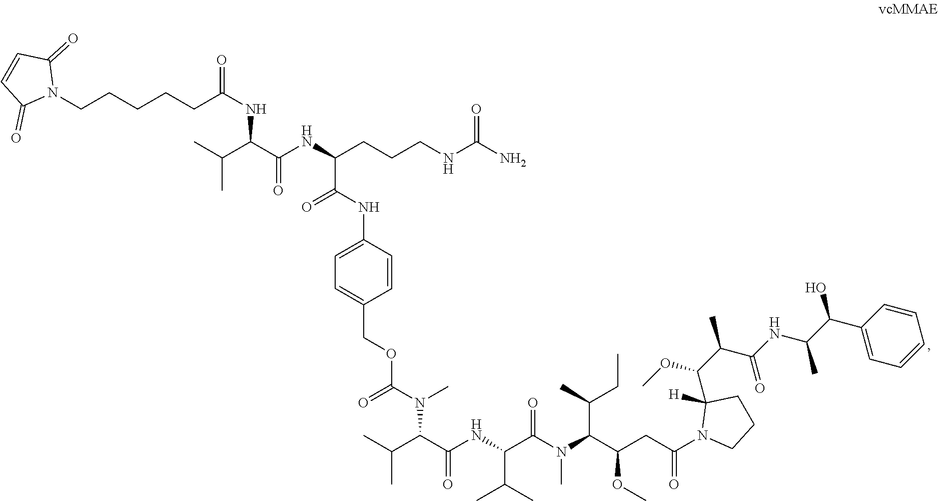 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

and wherein if the dose administered is greater than or equal to about 200 mg of the antibody or antigen-binding fragment thereof per treatment cycle, the method further comprises administering granulocyte colony stimulating factor (GCSF) to the subject is provided. In certain exemplary embodiments, if administered, the GCSF is administered prophylactically.

In another aspect, a method of treating a subject having or at risk of having a LIV-1-associated cancer, comprising administering to the subject granulocyte colony stimulating factor (GCSF), administering to the subject a therapeutically effective dose of an antibody or an antigen-binding fragment thereof that specifically binds human LIV-1, wherein the dose administered is greater than or equal to about 200 mg and less than or equal to about 250 mg of the antibody or antigen-binding fragment thereof per treatment cycle, wherein the antibody or antigen-binding fragment thereof comprises an HCVR having at least 95% identity to SEQ ID NO: 1, and an LCVR having at least 95% identity to SEQ ID NO: 2, and wherein the antibody or antigen-binding fragment thereof is conjugated to vcMMAE:

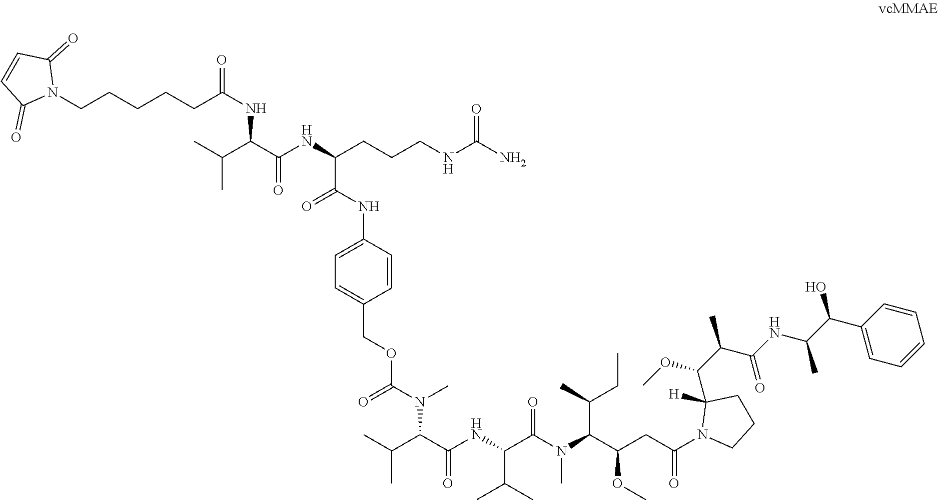 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

is provided. In certain exemplary embodiments, the GCSF is administered prophylactically.

In certain exemplary embodiments, the dose is administered at a concentration of about 2.5 mg/kg of body weight of the subject.

In certain exemplary embodiments, each treatment cycle is administered to the subject Q3W.

In certain exemplary embodiments, a vcMMAE to antibody or antigen-binding fragment thereof ratio is from about 1 to about 8 or about 4.

In certain exemplary embodiments, the LIV-1-associated cancer is a breast cancer, a triple negative breast cancer, a metastatic breast cancer, a triple-negative, metastatic breast cancer, or a hormone receptor-positive, metastatic breast cancer.

In certain exemplary embodiments, the subject is a human.

In another aspect, a method of treating a subject having or at risk of having a LIV-1-associated cancer, comprising administering to the subject a therapeutically effective dose of an antibody or an antigen-binding fragment thereof that specifically binds human LIV-1, wherein a dose administered is less than about 200 mg of the antibody or antigen-binding fragment thereof per Q3W treatment cycle, wherein the antibody or antigen-binding fragment thereof comprises an HCVR of SEQ ID NO: 1, and an LCVR of SEQ ID NO: 2, and wherein the antibody or antigen-binding fragment thereof is conjugated to vcMMAE:

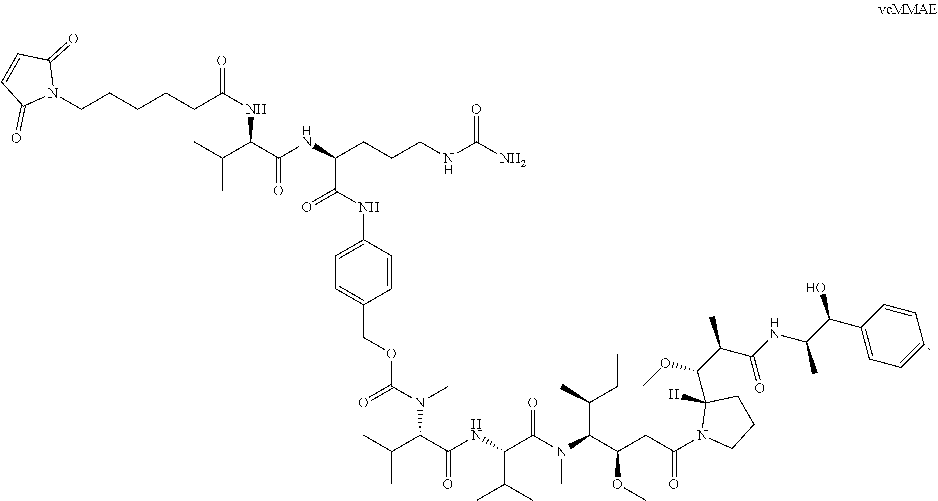 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

is provided.

In another aspect, a method of treating a subject having or at risk of having a LIV-1-associated cancer, comprising administering to the subject a therapeutically effective dose of an antibody or an antigen-binding fragment thereof that specifically binds human LIV-1, wherein a dose administered is less than or equal to about 250 mg of the antibody or antigen-binding fragment thereof per Q3W treatment cycle, wherein the antibody or antigen-binding fragment thereof comprises an HCVR of SEQ ID NO: 1, and an LCVR of SEQ ID NO: 2, wherein the antibody or antigen-binding fragment thereof is conjugated to vcMMAE:

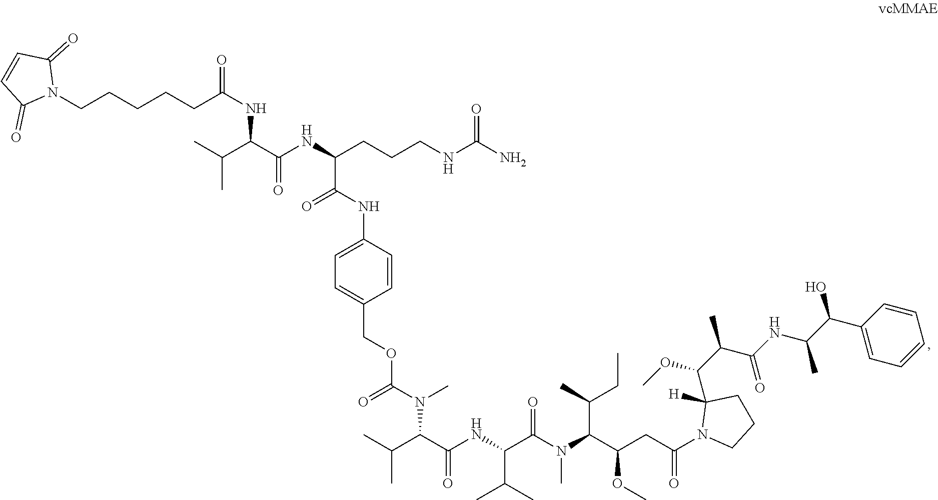 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

and wherein if the dose administered is greater than or equal to about 200 mg of the antibody or antigen-binding fragment thereof per treatment cycle, the method further comprises administering granulocyte colony stimulating factor (GCSF) to the subject is provided. In certain exemplary embodiments, if administered, the GCSF is administered prophylactically.

In another aspect, a method of treating a subject having or at risk of having a LIV-1-associated cancer, comprising administering to the subject granulocyte colony stimulating factor (GCSF), administering to the subject a therapeutically effective dose of an antibody or an antigen-binding fragment thereof that specifically binds human LIV-1, wherein a dose administered is greater than or equal to about 200 mg and less than or equal to about 250 mg of the antibody or antigen-binding fragment thereof per Q3W treatment cycle, wherein the antibody or antigen-binding fragment thereof comprises an HCVR of SEQ ID NO: 1, and an LCVR of SEQ ID NO: 2, and wherein the antibody or antigen-binding fragment thereof is conjugated to vcMMAE:

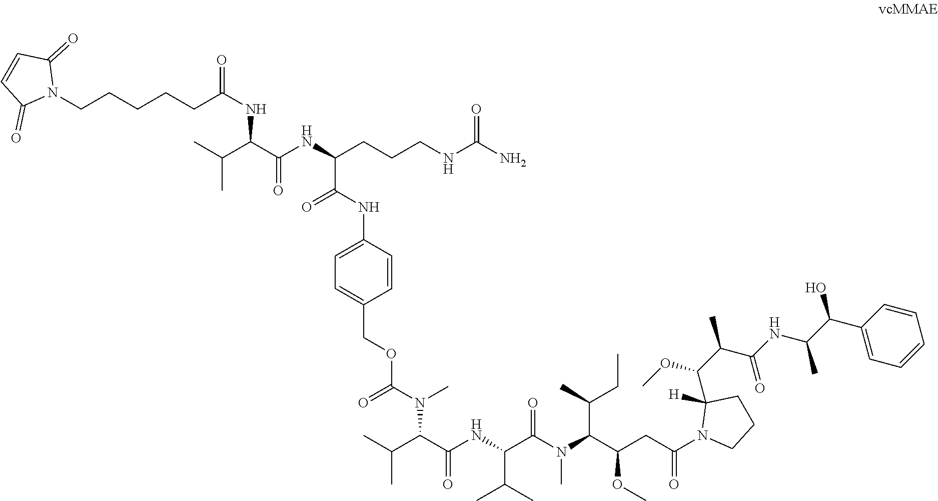 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

is provided. In certain exemplary embodiments, the GCSF is administered prophylactically.

In certain exemplary embodiments, the LIV-1-associated cancer is a breast cancer, a triple negative breast cancer, a metastatic breast cancer, a triple-negative, metastatic breast cancer, or a hormone receptor-positive, metastatic breast cancer.

In certain exemplary embodiments, a vcMMAE to antibody or antigen-binding fragment thereof ratio is about 4.

In certain exemplary embodiments, the dose is about 2.5 mg/kg of body weight of the subject.

In certain exemplary embodiments, the subject is a human.

In another aspect, a method of treating a subject having or at risk of having a LIV-1-associated breast cancer, comprising administering to the subject a dose of about 2.5 mg/kg of body weight of the subject an antibody or an antigen-binding fragment thereof that specifically binds human LIV-1, wherein the dose administered is less than about 200 mg of the antibody or antigen-binding fragment thereof per Q3W treatment cycle, wherein the antibody or antigen-binding fragment thereof comprises an HCVR of SEQ ID NO: 1, and an LCVR of SEQ ID NO: 2, and wherein the antibody or antigen-binding fragment thereof is conjugated to vcMMAE:

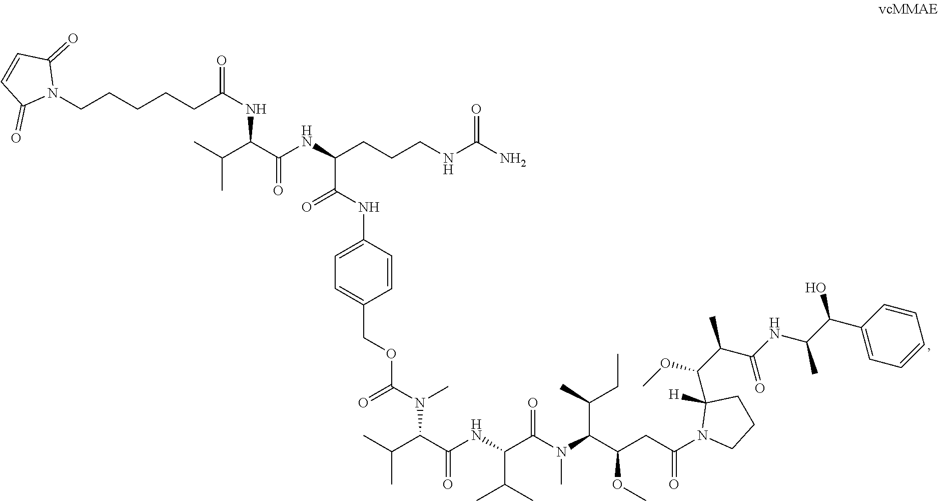 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

is provided.

In another aspect, a method of treating a subject having or at risk of having a LIV-1-associated breast cancer, comprising administering to the subject a dose of about 2.5 mg/kg of body weight of the subject an antibody or an antigen-binding fragment thereof that specifically binds human LIV-1, wherein the dose administered is less than or equal to about 250 mg of the antibody or antigen-binding fragment thereof per Q3W treatment cycle, wherein the antibody or antigen-binding fragment thereof comprises an HCVR of SEQ ID NO: 1, and an LCVR of SEQ ID NO: 2, wherein the antibody or antigen-binding fragment thereof is conjugated to vcMMAE:

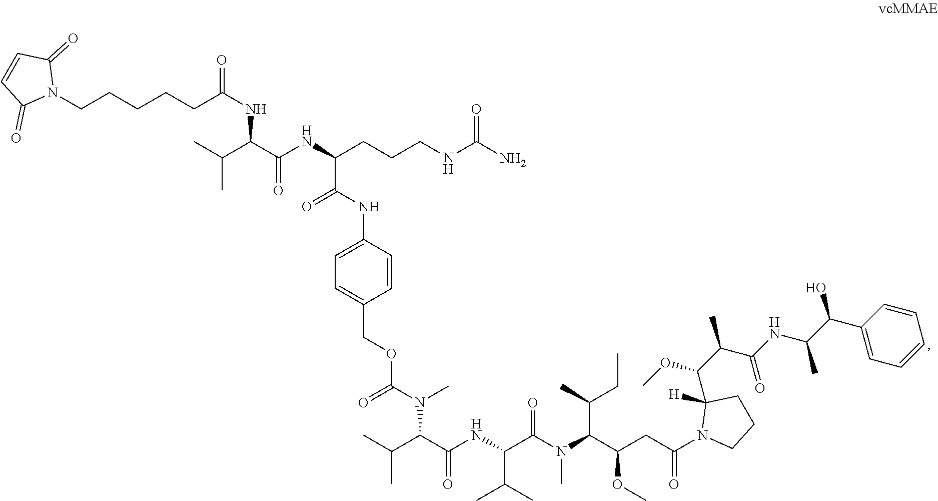 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

and wherein if the dose administered is greater than or equal to about 200 mg of the antibody or antigen-binding fragment thereof per treatment cycle, the method further comprises administering granulocyte colony stimulating factor (GCSF) to the subject is provided. In certain exemplary embodiments, if administered, the GCSF is administered prophylactically.

In another aspect, a method of treating a subject having or at risk of having a LIV-1-associated breast cancer, comprising administering to the subject granulocyte colony stimulating factor (GCSF), administering to the subject a dose of about 2.5 mg/kg of body weight of the subject an antibody or an antigen-binding fragment thereof that specifically binds human LIV-1, wherein the dose administered is greater than or equal to about 200 mg and less than or equal to about 250 mg of the antibody or antigen-binding fragment thereof per Q3W treatment cycle, wherein the antibody or antigen-binding fragment thereof comprises an HCVR of SEQ ID NO: 1, and an LCVR of SEQ ID NO: 2, and wherein the antibody or antigen-binding fragment thereof is conjugated to vcMMAE:

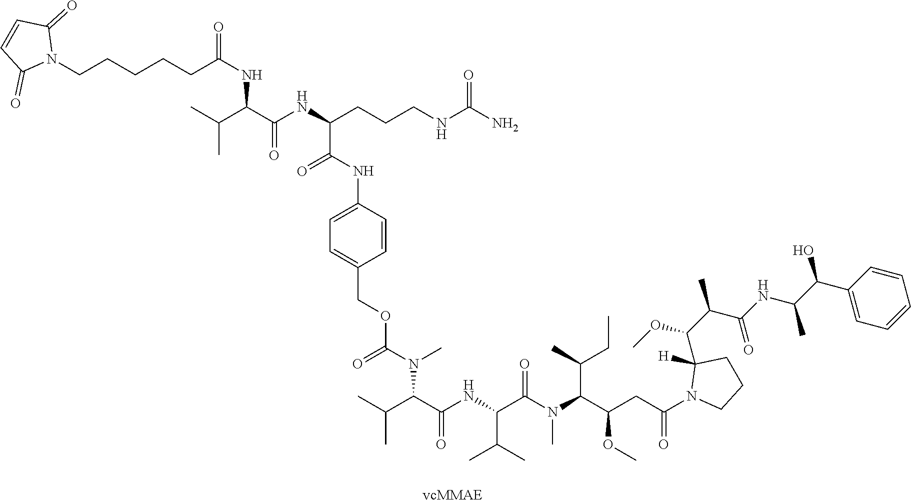 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

is provided. In certain exemplary embodiments, the GCSF is administered prophylactically.

In certain exemplary embodiments, the breast cancer is a triple negative breast cancer, a metastatic breast cancer, a triple-negative, metastatic breast cancer, or a hormone receptor-positive, metastatic breast cancer.

In certain exemplary embodiments, a vcMMAE to antibody or antigen-binding fragment thereof ratio is about 4.

In certain exemplary embodiments, the subject is a human.

The summary of the disclosure described above is non-limiting, and other features and advantages of the disclosed antibodies and methods of making and using them will be apparent from the detailed description, the example and the claims.

### DETAILED DESCRIPTION

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

### I. Definitions

As used herein, including the appended claims, the singular forms of words such as “a,” “an,” and “the,” include their corresponding plural references unless the context clearly dictates otherwise.

An “antibody-drug conjugate” or “ADC” refers to an antibody conjugated to a cytotoxic agent or cytostatic agent. Typically, antibody-drug conjugates bind to a target antigen (e.g., LIV1) on a cell surface, followed by internalization of the antibody-drug conjugate into the cell and subsequent release of the drug into the cell. In certain exemplary embodiments, an antibody-drug conjugate is a LIV1-ADC.

A “polypeptide” or “polypeptide chain” is a polymer of amino acid residues joined by peptide bonds, whether produced naturally or synthetically. Polypeptides of less than about 10 amino acid residues are commonly referred to as “peptides.”

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures. Substituents such as carbohydrate groups are generally not specified, but may be present nonetheless.

The terms “amino-terminal” and “carboxy-terminal” denote positions within polypeptides. Where the context allows, these terms are used with reference to a particular sequence or portion of a polypeptide to denote proximity or relative position. For example, a certain sequence positioned carboxy-terminal to a reference sequence within a polypeptide is located proximal to the carboxy terminus of the reference sequence, but is not necessarily at the carboxy terminus of the complete polypeptide.

For purposes of classifying amino acids substitutions as conservative or nonconservative, the following amino acid substitutions are considered conservative substitutions: serine substituted by threonine, alanine, or asparagine; threonine substituted by proline or serine; asparagine substituted by aspartic acid, histidine, or serine; aspartic acid substituted by glutamic acid or asparagine; glutamic acid substituted by glutamine, lysine, or aspartic acid; glutamine substituted by arginine, lysine, or glutamic acid; histidine substituted by tyrosine or asparagine; arginine substituted by lysine or glutamine; methionine substituted by isoleucine, leucine or valine; isoleucine substituted by leucine, valine, or methionine; leucine substituted by valine, isoleucine, or methionine; phenylalanine substituted by tyrosine or tryptophan; tyrosine substituted by tryptophan, histidine, or phenylalanine; proline substituted by threonine; alanine substituted by serine; lysine substituted by glutamic acid, glutamine, or arginine; valine substituted by methionine, isoleucine, or leucine; and tryptophan substituted by phenylalanine or tyrosine. Conservative substitutions can also mean substitutions between amino acids in the same class. Classes are as follows: Group I (hydrophobic side chains): met, ala, val, leu, ile; Group II (neutral hydrophilic side chains): cys, ser, thr; Group III (acidic side chains): asp, glu; Group IV (basic side chains): asn, gin, his, lys, arg; Group V (residues influencing chain orientation): gly, pro; and Group VI (aromatic side chains): trp, tyr, phe.

Two amino acid sequences have “100% amino acid sequence identity” if the amino acid residues of the two amino acid sequences are the same when aligned for maximal correspondence. Sequence comparisons can be performed using standard software programs such as those included in the LASERGENE bioinformatics computing suite, which is produced by DNASTAR (Madison, Wis.). Other methods for comparing two nucleotide or amino acid sequences by determining optimal alignment are well-known to those of skill in the art. (See, e.g., Peruski and Peruski, The Internet and the New Biology: Tools for Genomic and Molecular Research (ASM Press, Inc. 1997); Wu et al. (eds.), “Information Superhighway and Computer Databases of Nucleic Acids and Proteins,” in Methods in Gene Biotechnology 123-151 (CRC Press, Inc. 1997); Bishop (ed.), Guide to Human Genome Computing (2nd ed., Academic Press, Inc. 1998).) Two amino acid sequences are considered to have “substantial sequence identity” if the two sequences have at least about 80%, at least about 85%, at about least 90%, or at least about 95% sequence identity relative to each other.

Percentage sequence identities are determined with antibody sequences maximally aligned by the Kabat numbering convention. After alignment, if a subject antibody region (e.g., the entire variable domain of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” one or more recited elements may include other elements not specifically recited. For example, a composition that comprises antibody may contain the antibody alone or in combination with other ingredients.

Designation of a range of values includes all integers within or defining the range.

In antibodies or other proteins described herein, reference to amino acid residues corresponding to those specified by SEQ ID NO includes post-translational modifications of such residues.

The term “antibody” denotes immunoglobulin proteins produced by the body in response to the presence of an antigen and that bind to the antigen, as well as antigen-binding fragments and engineered variants thereof. Hence, the term “antibody” includes, for example, intact monoclonal antibodies (e.g., antibodies produced using hybridoma technology) and antigen-binding antibody fragments, such as a F(ab′)₂, a Fv fragment, a diabody, a single-chain antibody, an scFv fragment, or an scFv-Fc. Genetically, engineered intact antibodies and fragments such as chimeric antibodies, humanized antibodies, single-chain Fv fragments, single-chain antibodies, diabodies, minibodies, linear antibodies, multivalent or multi-specific (e.g., bispecific) hybrid antibodies, and the like, are also included. Thus, the term “antibody” is used expansively to include any protein that comprises an antigen-binding site of an antibody and is capable of specifically binding to its antigen.

The term antibody or antigen-binding fragment thereof includes a “conjugated” antibody or antigen-binding fragment thereof or an “antibody-drug conjugate (ADC)” in which an antibody or antigen-binding fragment thereof is covalently or non-covalently bound to a pharmaceutical agent, e.g., to a cytostatic or cytotoxic drug.

The term “genetically engineered antibodies” refers to an antibody in which the amino acid sequence has been varied from that of the native or parental antibody. The possible variations are many, and range from the changing of just one or a few amino acids to the complete redesign of, for example, the variable or constant region. Changes in the constant region are, in general, made to improve or alter characteristics such as, e.g., complement binding and other effector functions. Typically, changes in the variable region are made to improve antigen-binding characteristics, improve variable region stability, and/or reduce the risk of immunogenicity.

The term “chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

An “antigen-binding site of an antibody” is that portion of an antibody that is sufficient to bind to its antigen. The minimum such region is typically a variable domain or a genetically engineered variant thereof. Single domain binding sites can be generated from camelid antibodies (see Muyldermans and Lauwereys, Mol. Recog. 12: 131-140, 1999; Nguyen et al., EMBO J. 19:921-930, 2000) or from VH domains of other species to produce single-domain antibodies (“dAbs,” see Ward et al., Nature 341: 544-546, 1989; U.S. Pat. No. 6,248,516 to Winter et al). Commonly, an antigen-binding site of an antibody comprises both a heavy chain variable (VH) domain and a light chain variable (VL) domain that bind to a common epitope. Within the context of the present invention, an antibody may include one or more components in addition to an antigen-binding site, such as, for example, a second antigen-binding site of an antibody (which may bind to the same or a different epitope or to the same or a different antigen), a peptide linker, an immunoglobulin constant region, an immunoglobulin hinge, an amphipathic helix (see Pack and Pluckthun, Biochem. 31: 1579-1584, 1992), a non-peptide linker, an oligonucleotide (see Chaudri et al., FEBS Letters 450:23-26, 1999), a cytostatic or cytotoxic drug, and the like, and may be a monomeric or multimeric protein. Examples of molecules comprising an antigen-binding site of an antibody are known in the art and include, for example, Fv, single-chain Fv (scFv), Fab, Fab′, F(ab′)2, F(ab)c, diabodies, minibodies, nanobodies, Fab-scFv fusions, bispecific (scFv)4-IgG, and bispecific (scFv)2-Fab. (See, e.g., Hu et al, Cancer Res. 56:3055-3061, 1996; Atwell et al., Molecular Immunology 33: 1301-1312, 1996; Carter and Merchant, Curr. Op. Biotechnol. 8:449-454, 1997; Zuo et al., Protein Engineering 13:361-367, 2000; and Lu et al., J. Immunol. Methods 267:213-226, 2002.)

The term “immunoglobulin” refers to a protein consisting of one or more polypeptides substantially encoded by immunoglobulin gene(s). One form of immunoglobulin constitutes the basic structural unit of native (i.e., natural or parental) antibodies in vertebrates. This form is a tetramer and consists of two identical pairs of immunoglobulin chains, each pair having one light chain and one heavy chain. In each pair, the light and heavy chain variable regions (VL and VH) are together primarily responsible for binding to an antigen, and the constant regions are primarily responsible for the antibody effector functions. Five classes of immunoglobulin protein (IgG, IgA, IgM, IgD, and IgE) have been identified in higher vertebrates. IgG comprises the major class, and it normally exists as the second most abundant protein found in plasma. In humans, IgG consists of four subclasses, designated IgG1, IgG2, IgG3, and IgG4. Each immunoglobulin heavy chain possesses a constant region that consists of constant region protein domains (CHL hinge, CH2, and CH3; IgG3 also contains a CH4 domain) that are essentially invariant for a given subclass in a species.

DNA sequences encoding human and non-human immunoglobulin chains are known in the art. (See, e.g., Ellison et al, DNA 1: 11-18, 1981; Ellison et al, Nucleic Acids Res. 10:4071-4079, 1982; Kenten et al., Proc. Natl. Acad. Set USA 79:6661-6665, 1982; Seno et al., Nucl. Acids Res. 11:719-726, 1983; Riechmann et al., Nature 332:323-327, 1988; Amster et al., Nucl. Acids Res. 8:2055-2065, 1980; Rusconi and Kohler, Nature 314:330-334, 1985; Boss et al., Nucl. Acids Res. 12:3791-3806, 1984; Bothwell et al., Nature 298:380-382, 1982; van der Loo et al., Immunogenetics 42:333-341, 1995; Karlin et al., J. Mol. Evol. 22: 195-208, 1985; Kindsvogel et al., DNA 1:335-343, 1982; Breiner et al., Gene 18: 165-174, 1982; Kondo et al., Eur. J. Immunol. 23:245-249, 1993; and GenBank Accession No. J00228.) For a review of immunoglobulin structure and function see Putnam, The Plasma Proteins, Vol V, Academic Press, Inc., 49-140, 1987; and Padlan, Mol. Immunol. 31: 169-217, 1994. The term “immunoglobulin” is used herein for its common meaning, denoting an intact antibody, its component chains, or fragments of chains, depending on the context.

Full-length immunoglobulin “light chains” (about 25 kDa or 214 amino acids) are encoded by a variable region gene at the amino-terminus (encoding about 110 amino acids) and a by a kappa or lambda constant region gene at the carboxyl-terminus. Full-length immunoglobulin “heavy chains” (about 50 kDa or 446 amino acids) are encoded by a variable region gene (encoding about 116 amino acids) and a gamma, mu, alpha, delta, or epsilon constant region gene (encoding about 330 amino acids), the latter defining the antibody's isotype as IgG, IgM, IgA, IgD, or IgE, respectively. Within light and heavy chains, the variable and constant regions are joined by a “J” region of about 12 or more amino acids, with the heavy chain also including a “D” region of about 10 more amino acids. (See generally Fundamental Immunology (Paul, ed., Raven Press, N.Y., 2nd ed. 1989), Ch. 7).

An immunoglobulin light or heavy chain variable region (also referred to herein as a “light chain variable domain” (“VL domain”) or “heavy chain variable domain” (“VH domain”), respectively) consists of a “framework” region interrupted by three “complementarity determining regions” or “CDRs.” The framework regions serve to align the CDRs for specific binding to an epitope of an antigen. Thus, the term “CDR” refers to the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, FR4.

The assignment of amino acids to each variable region domain is in accordance with the definitions of Kabat, Sequences of Proteins of Immunological Interest (National Institutes of Health, Bethesda, Md., 1987 and 1991). Kabat also provides a widely used numbering convention (Kabat numbering) in which corresponding residues between different heavy chain variable regions or between different light chain variable regions are assigned the same number. CDRs 1, 2 and 3 of a VL domain are also referred to herein, respectively, as CDR-L1, CDR-L2 and CDR-L3. CDRs 1, 2 and 3 of a VH domain are also referred to herein, respectively, as CDR-H1, CDR-H2 and CDR-H3. If so noted, the assignment of CDRs can be in accordance with IMGT® (Lefranc et al., Developmental & Comparative Immunology 27:55-77; 2003) in lieu of Kabat.

Numbering of the heavy chain constant region is via the EU index as set forth in Kabat (Kabat, Sequences of Proteins of Immunological Interest, National Institutes of Health, Bethesda, Md., 1987 and 1991).

Unless the context dictates otherwise, the term “monoclonal antibody” is not limited to antibodies produced through hybridoma technology. The term “monoclonal antibody” can include an antibody that is derived from a single clone, including any eukaryotic, prokaryotic or phage clone. In particular embodiments, the antibodies described herein are monoclonal antibodies.

The term “humanized VH domain” or “humanized VL domain” refers to an immunoglobulin VH or VL domain comprising some or all CDRs entirely or substantially from a non-human donor immunoglobulin (e.g., a mouse or rat) and variable domain framework sequences entirely or substantially from human immunoglobulin sequences. The non-human immunoglobulin providing the CDRs is called the “donor” and the human immunoglobulin providing the framework is called the “acceptor.” In some instances, humanized antibodies will retain some non-human residues within the human variable domain framework regions to enhance proper binding characteristics (e.g., mutations in the frameworks may be required to preserve binding affinity when an antibody is humanized).

A “humanized antibody” is an antibody comprising one or both of a humanized VH domain and a humanized VL domain. Immunoglobulin constant region(s) need not be present, but if they are, they are entirely or substantially from human immunoglobulin constant regions.

A humanized antibody is a genetically engineered antibody in which the CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557). The acceptor antibody sequences can be, for example, a mature human antibody sequence, a composite of such sequences, a consensus sequence of human antibody sequences, or a germline region sequence.

Human acceptor sequences can be selected for a high degree of sequence identity in the variable region frameworks with donor sequences to match canonical forms between acceptor and donor CDRs among other criteria. Thus, a humanized antibody is an antibody having CDRs entirely or substantially from a donor antibody and variable region framework sequences and constant regions, if present, entirely or substantially from human antibody sequences. Similarly, a humanized heavy chain typically has all three CDRs entirely or substantially from a donor antibody heavy chain, and a heavy chain variable region framework sequence and heavy chain constant region, if present, substantially from human heavy chain variable region framework and constant region sequences. Similarly, a humanized light chain typically has all three CDRs entirely or substantially from a donor antibody light chain, and a light chain variable region framework sequence and light chain constant region, if present, substantially from human light chain variable region framework and constant region sequences.

A CDR in a humanized antibody is substantially from a corresponding CDR in a non-human antibody when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering), or wherein about 100% of corresponding residues (as defined by Kabat numbering), are identical between the respective CDRs. The variable region framework sequences of an antibody chain or the constant region of an antibody chain are substantially from a human variable region framework sequence or human constant region respectively when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering for the variable region and EU numbering for the constant region), or about 100% of corresponding residues (as defined by Kabat numbering for the variable region and EU numbering for the constant region) are identical.

Although humanized antibodies often incorporate all six CDRs (preferably as defined by Kabat or IMGT®) from a mouse antibody, they can also be made with fewer than all six CDRs (e.g., at least 3, 4, or 5) CDRs from a mouse antibody (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164: 1432-1441, 2000).

A CDR in a humanized antibody is “substantially from” a corresponding CDR in a non-human antibody when at least 60%, at least 85%, at least 90%, at least 95% or 100% of corresponding residues (as defined by Kabat (or IMGT)) are identical between the respective CDRs. In particular variations of a humanized VH or VL domain in which CDRs are substantially from a non-human immunoglobulin, the CDRs of the humanized VH or VL domain have no more than six (e.g., no more than five, no more than four, no more than three, no more than two, or nor more than one) amino acid substitutions (preferably conservative substitutions) across all three CDRs relative to the corresponding non-human VH or VL CDRs. The variable region framework sequences of an antibody VH or VL domain or, if present, a sequence of an immunoglobulin constant region, are “substantially from” a human VH or VL framework sequence or human constant region, respectively, when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering for the variable region and EU numbering for the constant region), or about 100% of corresponding residues (as defined by Kabat numbering for the variable region and EU numbering for the constant region) are identical. Hence, all parts of a humanized antibody, except the CDRs, are typically entirely or substantially from corresponding parts of natural human immunoglobulin sequences.

Antibodies are typically provided in isolated form. This means that an antibody is typically at least about 50% w/w pure of interfering proteins and other contaminants arising from its production or purification but does not exclude the possibility that the antibody is combined with an excess of pharmaceutical acceptable carrier(s) or other vehicle intended to facilitate its use. Sometimes antibodies are at least about 60%, about 70%, about 80%, about 90%, about 95% or about 99% w/w pure of interfering proteins and contaminants from production or purification. Antibodies, including isolated antibodies, can be conjugated to cytotoxic agents and provided as antibody drug conjugates.

Specific binding of an antibody to its target antigen typically refers an affinity of at least about 10⁶, about 10⁷, about 10⁸, about 10⁹, or about 10¹⁰ M⁻¹. Specific binding is detectably higher in magnitude and distinguishable from non-specific binding occurring to at least one non-specific target. Specific binding can be the result of formation of bonds between particular functional groups or particular spatial fit (e.g., lock and key type), whereas nonspecific binding is typically the result of van der Waals forces.

The term “epitope” refers to a site of an antigen to which an antibody binds. An epitope can be formed from contiguous amino acids or noncontiguous amino acids juxtaposed by tertiary folding of one or more proteins. Epitopes formed from contiguous amino acids are typically retained upon exposure to denaturing agents, e.g., solvents, whereas epitopes formed by tertiary folding are typically lost upon treatment with denaturing agents, e.g., solvents. An epitope typically includes at least about 3, and more usually, at least about 5, at least about 6, at least about 7, or about 8-10 amino acids in a unique spatial conformation. Methods of determining spatial conformation of epitopes include, for example, x-ray crystallography and two-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody bound to its antigen to identify contact residues.

Alternatively, two antibodies have the same epitope if all amino acid mutations in the antigen that reduce or eliminate binding of one antibody reduce or eliminate binding of the other (provided that such mutations do not produce a global alteration in antigen structure). Two antibodies have overlapping epitopes if some amino acid mutations that reduce or eliminate binding of one antibody reduce or eliminate binding of the other antibody.

Competition between antibodies can be determined by an assay in which a test antibody inhibits specific binding of a reference antibody to a common antigen (see, e.g., Junghans et al., Cancer Res. 50: 1495, 1990). A test antibody competes with a reference antibody if an excess of a test antibody inhibits binding of the reference antibody.

Antibodies identified by competition assay (competing antibodies) include antibodies that bind to the same epitope as the reference antibody and antibodies that bind to an adjacent epitope sufficiently proximal to the epitope bound by the reference antibody for steric hindrance to occur. Antibodies identified by a competition assay also include those that indirectly compete with a reference antibody by causing a conformational change in the target protein thereby preventing binding of the reference antibody to a different epitope than that bound by the test antibody.

An antibody effector function refers to a function contributed by an Fc region of an Ig. Such functions can be, for example, antibody-dependent cellular cytotoxicity (ADCC), antibody-dependent cellular phagocytosis (ADCP), or complement-dependent cytotoxicity (CDC). Such function can be affected by, for example, binding of an Fc region to an Fc receptor on an immune cell with phagocytic or lytic activity or by binding of an Fc region to components of the complement system. Typically, the effect(s) mediated by the Fc-binding cells or complement components result in inhibition and/or depletion of the LIV1-targeted cell. Fc regions of antibodies can recruit Fc receptor (FcR)-expressing cells and juxtapose them with antibody-coated target cells. Cells expressing surface FcR for IgGs including FcγRIII (CD16), FcγRII (CD32) and FcγRIII (CD64) can act as effector cells for the destruction of IgG-coated cells. Such effector cells include monocytes, macrophages, natural killer (NK) cells, neutrophils and eosinophils. Engagement of FcγR by IgG activates ADCC or ADCP. ADCC is mediated by CD16+ effector cells through the secretion of membrane pore-forming proteins and proteases, while phagocytosis is mediated by CD32+ and CD64+ effector cells (see Fundamental Immunology, 4^{th} ed., Paul ed., Lippincott-Raven, N.Y., 1997, Chapters 3, 17 and 30; Uchida et al., J. Exp. Med. 199:1659-69, 2004; Akewanlop et al., Cancer Res. 61:4061-65, 2001; Watanabe et al., Breast Cancer Res. Treat. 53: 199-207, 1999).

In addition to ADCC and ADCP, Fc regions of cell-bound antibodies can also activate the complement classical pathway to elicit CDC. C1q of the complement system binds to the Fc regions of antibodies when they are complexed with antigens. Binding of C1q to cell-bound antibodies can initiate a cascade of events involving the proteolytic activation of C4 and C2 to generate the C3 convertase. Cleavage of C3 to C3b by C3 convertase enables the activation of terminal complement components including C5b, C6, C7, C8 and C9. Collectively, these proteins form membrane-attack complex pores on the antibody-coated cells. These pores disrupt the cell membrane integrity, killing the target cell (see Immunobiology, 6^{th} ed., Janeway et al, Garland Science, N.Y., 2005, Chapter 2).

The term “antibody-dependent cellular cytotoxicity” or “ADCC” refers to a mechanism for inducing cell death that depends on the interaction of antibody-coated target cells with immune cells possessing lytic activity (also referred to as effector cells). Such effector cells include natural killer cells, monocytes/macrophages and neutrophils. The effector cells attach to an Fc region of Ig bound to target cells via their antigen-combining sites. Death of the antibody-coated target cell occurs as a result of effector cell activity. In certain exemplary embodiments, an anti-LIV1 IgG1 antibody of the invention mediates equal or increased ADCC relative to a parental antibody and/or relative to an anti-LIV1 IgG3 antibody.

The term “antibody-dependent cellular phagocytosis” or “ADCP” refers to the process by which antibody-coated cells are internalized, either in whole or in part, by phagocytic immune cells (e.g., by macrophages, neutrophils and/or dendritic cells) that bind to an Fc region of Ig. In certain exemplary embodiments, an anti-LIV1 IgG1 antibody of the invention mediates equal or increased ADCP relative to a parental antibody and/or relative to an anti-LIV1 IgG3 antibody.

The term “complement-dependent cytotoxicity” or “CDC” refers to a mechanism for inducing cell death in which an Fc region of a target-bound antibody activates a series of enzymatic reactions culminating in the formation of holes in the target cell membrane.

Typically, antigen-antibody complexes such as those on antibody-coated target cells bind and activate complement component C1q, which in turn activates the complement cascade leading to target cell death. Activation of complement may also result in deposition of complement components on the target cell surface that facilitate ADCC by binding complement receptors (e.g., CR3) on leukocytes.

A “cytotoxic effect” refers to the depletion, elimination and/or killing of a target cell. A “cytotoxic agent” refers to a compound that has a cytotoxic effect on a cell, thereby mediating depletion, elimination and/or killing of a target cell. In certain embodiments, a cytotoxic agent is conjugated to an antibody or administered in combination with an antibody. Suitable cytotoxic agents are described further herein.

A “cytostatic effect” refers to the inhibition of cell proliferation. A “cytostatic agent” refers to a compound that has a cytostatic effect on a cell, thereby mediating inhibition of growth and/or expansion of a specific cell type and/or subset of cells. Suitable cytostatic agents are described further herein.

The term “patient” or “subject” includes human and other mammalian subjects such as non-human primates, rabbits, rats, mice, and the like and transgenic species thereof, that receive either prophylactic or therapeutic treatment.

The term “effective amount,” in the context of treatment of a LIV1-expressing disorder by administration of an anti-LIV1 antibody or antigen-binding fragment thereof (e.g., a LIV1-ADC) as described herein, refers to an amount of such antibody or antigen-binding fragment thereof that is sufficient to inhibit the occurrence or ameliorate one or more symptoms of a LIV1-related disorder (e.g., a LIV1-expressing cancer). An effective amount of an antibody is administered in an “effective regimen.” The term “effective regimen” refers to a combination of amount of the antibody being administered and dosage frequency adequate to accomplish prophylactic or therapeutic treatment of the disorder (e.g., prophylactic or therapeutic treatment of a LIV1-expressing cancer).

The term “pharmaceutically acceptable” means approved or approvable by a regulatory agency of the Federal or a state government or listed in the U.S. Pharmacopeia or other generally recognized pharmacopeia for use in animals, and more particularly in humans. The term “pharmaceutically compatible ingredient” refers to a pharmaceutically acceptable diluent, adjuvant, excipient, or vehicle with which an anti-LIV1 antibody (e.g., a LIV1-ADC) is formulated.

The phrase “pharmaceutically acceptable salt,” refers to pharmaceutically acceptable organic or inorganic salts. Exemplary salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p toluenesulfonate, and pamoate (i.e., 1,1′-methylene bis-(2 hydroxy-3-naphthoate) salts. A pharmaceutically acceptable salt may further comprise an additional molecule such as, e.g., an acetate ion, a succinate ion or other counterion. A counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

Unless otherwise apparent from the context, when a value is expressed as “about” X or “approximately” X, the stated value of X will be understood to be accurate to ±10%.

Solvates in the context of the invention are those forms of the compounds of the invention that form a complex in the solid or liquid state through coordination with solvent molecules. Hydrates are one specific form of solvates, in which the coordination takes place with water. In certain exemplary embodiments, solvates in the context of the present invention are hydrates.

### II. Anti-LIV1 Antibodies and Antigen-Binding Fragments

The present invention provides isolated, recombinant and/or synthetic anti-LIV1 human, primate, rodent, mammalian, chimeric, humanized and/or CDR-grafted antibodies and antigen-binding fragments thereof (e.g., a LIV1-ADC), as well as compositions and nucleic acid molecules comprising at least one polynucleotide encoding at least a portion of one anti-LIV1 antibody molecule. The present invention further includes, but is not limited to, methods of making and using such nucleic acids and antibodies including diagnostic and therapeutic compositions, methods and devices. In certain exemplary embodiments, humanized anti-LIV1 IgG1 antibodies are provided. In other exemplary embodiments, humanized anti-LIV1 IgG1 antibody-drug conjugates are provided.

Unless otherwise indicated, an anti-LIV1-antibody drug conjugate (i.e., a LIV1-ADC) includes an antibody specific for the human LIV-1 protein conjugated to a cytotoxic agent.

SGN-LIV1A is an anti-LIV-1 humanized antibody (also referred to as hLIV22) which is conjugated to monomethyl auristatin E (MMAE) via a protease-cleavable linker (i.e., a valine-citrulline linker). Upon binding to a LIV-1 expressing cell, SGN-LIV1A is internalized and releases MMAE, which disrupts microtubulin and induces apoptosis.

SGN-LIV1A is a humanized form of the mouse BR2-22a antibody, described in U.S. Pat. No. 9,228,026. The SGN-LIV1A antibody is essentially the same as BR2-22a within experimental error and contains seven back mutations. Methods of making the SGN-LIV1A antibody are also disclosed in U.S. Pat. No. 9,228,026, which is incorporated herein by reference in its entirety for all purposes.

The amino acid sequence of the heavy chain variable region of SGN-LIV1A is provided herein as SEQ ID NO: 1. The amino acid sequence of the light chain variable region of SGN-LIV1A is provided herein as SEQ ID NO: 2. Synthesis and conjugation of the drug linker vcMMAE (shown below; also referred to as 1006) are further described in U.S. Pat. No. 9,228,026 and US Patent Pub. No. 2005/0238649, which are incorporated herein by reference in their entireties for all purposes.

TABLE 1
HCVR of SGN-LIV1A (SEQ ID NO: 1).

Gln Val Gln Leu Val Gln Ser Gly Ala Glu Val Lys
Lys Pro Gly Ala Ser Val Lys Val Ser Cys Lys Ala
Ser Gly Leu Thr Ile Glu Asp Tyr Tyr Met His Trp
Val Arg Gln Ala Pro Gly Gln Gly Leu Glu Trp Met
Gly Trp Ile Asp Pro Glu Asn Gly Asp Thr Glu Tyr
Gly Pro Lys Phe Gln Gly Arg Val Thr Met Thr Arg
Asp Thr Ser Ile Asn Thr Ala Tyr Met Glu Leu Ser
Arg Leu Arg Ser Asp Asp Thr Ala Val Tyr Tyr Cys
Ala Val His Asn Ala His Tyr Gly Thr Trp Phe Ala
Tyr Trp Gly Gln Gly Thr Leu Val Thr Val Ser Ser

TABLE 2
LCVR of SGN-LIV1A (SEQ ID NO: 2).

Asp Val Val Met Thr Gln Ser Pro Leu Ser Leu Pro
Val Thr Leu Gly Gln Pro Ala Ser Ile Ser Cys Arg
Ser Ser Gln Ser Leu Leu His Ser Ser Gly Asn Thr
Tyr Leu Glu Trp Tyr Gln Gln Arg Pro Gly Gln Ser
Pro Arg Pro Leu Ile Tyr Lys Ile Ser Thr Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Gly
Ser His Val Pro Tyr Thr Phe Gly Gly Gly Thr Lys
Val Glu Ile Lys Arg

According to certain exemplary embodiments, a LIV1-ADC comprises monomethyl auristatin E (MMAE) (PubChem CID: 53297465):

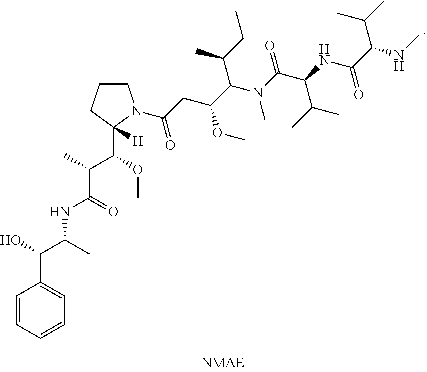 [SMILES: CC[C@H](C)C([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

According to certain exemplary embodiments, a LIV1-ADC comprises vcMMAE conjugated thereto. vcMMAE is a drug-linker conjugate for ADC with potent anti-tumor activity comprising the anti-mitotic agent, MMAE, linked via the lysosomally cleavable dipeptide valine-citrulline (vc):

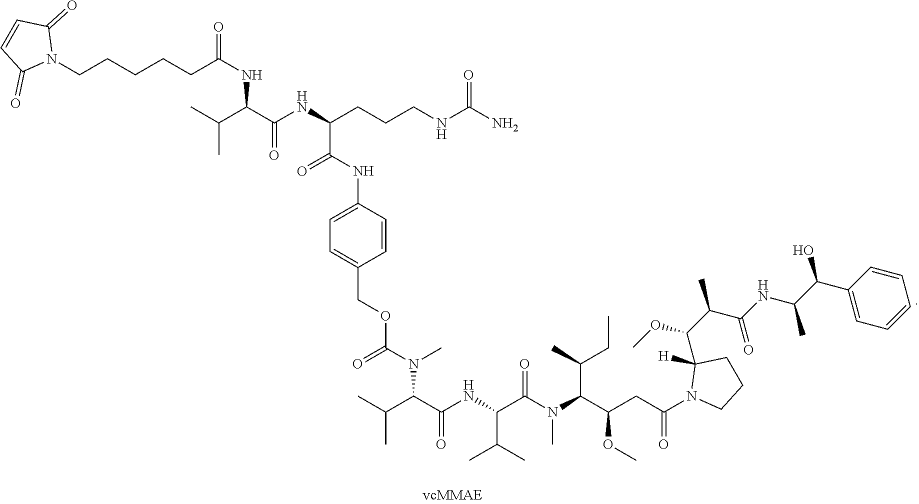 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

U.S. Pat. No. 9,228,026 discloses methods for conjugating vcMMAE to hLIV22.

A vcMMAE-antibody conjugate (e.g., a LIV1-ADC) according to certain exemplary embodiments is set forth below.

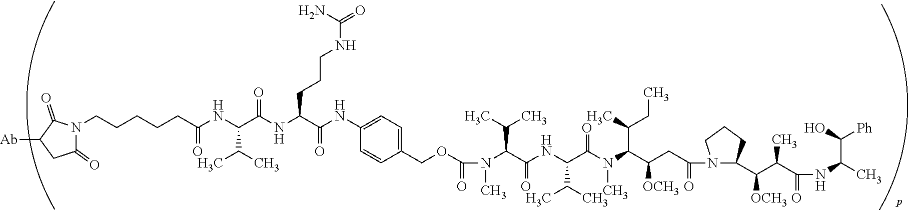

According to certain exemplary embodiments, a vcMMAE-antibody conjugate (e.g., a LIV1-ADC) is provided as set forth above, wherein Ab may include an anti-LIV1 antibody or antigen-binding fragment thereof (e.g., hLIV22), and wherein p may be any integer from about 1 to about 8. In some embodiments, a vcMMAE-antibody conjugate (e.g., a LIV1-ADC) is provided as set forth above, wherein Ab may include an anti-LIV1 antibody or antigen-binding fragment thereof (e.g., hLIV22), and wherein p is 1, representing a vcMMAE to antibody or antigen-binding fragment thereof ratio of 1. In some embodiments, a vcMMAE-antibody conjugate (e.g., a LIV1-ADC) is provided as set forth above, wherein Ab may include an anti-LIV1 antibody or antigen-binding fragment thereof (e.g., hLIV22), and wherein p is 2, 3, 4, 5, 6, 7, 8, 9, or 10, representing a vcMMAE to antibody or antigen-binding fragment thereof ratio (also known as a “Drug-to-Antibody Ratio” or “DAR”) of 2, 3, 4, 5, 6, 7, 8, 9, or 10, respectively. Accordingly, in some embodiments, a vcMMAE-antibody conjugate (e.g., a LIV1-ADC) is provided as set forth above, wherein a vcMMAE to antibody or antigen-binding fragment thereof ratio is 1, 2, 3, 4, 5, 6, 7, 8, 9, or 10. In certain exemplary embodiments, a vcMMAE-antibody conjugate (e.g., a LIV1-ADC) is provided as set forth above, wherein Ab may include an anti-LIV1 antibody or antigen-binding fragment thereof (e.g., hLIV22), and wherein p is 4, representing a vcMMAE to antibody or antigen-binding fragment thereof ratio of 4. Accordingly, in certain exemplary embodiments, a vcMMAE-antibody conjugate (e.g., a LIV1-ADC) is provided as set forth above, wherein a vcMMAE to antibody or antigen-binding fragment thereof ratio is 4.

SGN-LIV1A can be administered to subjects at a level that inhibits breast cancer cell growth, while at the same time is tolerated by the subject.

In certain exemplary embodiments, an anti-LIV1 antibody or antigen-binding fragment thereof (e.g., a LIV1-ADC) comprises CDRs from an HCVR set forth as SEQ ID NO: 1 and/or CDRs from an LCVR set forth as SEQ ID NO: 2. In certain exemplary embodiments, an anti-LIV1 antibody or antigen-binding fragment thereof (e.g., a LIV1-ADC) comprises an HCVR set forth as SEQ ID NO: 1 and/or an LCVR set forth as SEQ ID NO: 2. In other embodiments, an anti-LIV1 antibody or antigen-binding fragment thereof (e.g., a LIV1-ADC) comprises an HCVR/LCVR pair SEQ ID NO: 1/SEQ ID NO: 2. In other embodiments, an anti-LIV1 antibody or antigen-binding fragment thereof (e.g., a LIV1-ADC) comprises an HCVR that has at least about 80% homology or identity (e.g., 80%, 85%, 90%, 91%, 92%, 93%, 94%, 95%, 96%, 97%, 98% or 99%) to SEQ ID NO: 1 and/or comprises an LCVR that has at least about 80% homology or identity (e.g., 80%, 85%, 90%, 91%, 92%, 93%, 94%, 95%, 96%, 97%, 98% or 99%) to SEQ ID NO: 2.

Anti-LIV1 antibodies and antigen-binding fragments thereof (e.g., LIV1-ADCs) described herein can be expressed in a modified form. For instance, a region of additional amino acids, particularly charged amino acids, can be added to the N-terminus of an anti-LIV1 antibody or an antigen-binding fragment thereof (e.g., a LIV1-ADC) to improve stability and persistence in the host cell, during purification, or during subsequent handling and storage. Also, peptide moieties can be added to an anti-LIV1 antibody or an antigen-binding fragment thereof (e.g., a LIV1-ADC) of the present invention to facilitate purification. Such regions can be removed prior to final preparation of an antibody molecule or at least one fragment thereof. Such methods are described in many standard laboratory manuals, such as Sambrook, supra; Ausubel, et al., ed., Current Protocols In Molecular Biology, John Wiley & Sons, Inc., NY, N.Y. (1987-2001).

The anti-LIV1 antibodies or antigen-binding fragments thereof (e.g., LIV1-ADCs) described herein typically bind LIV-1 with an equilibrium binding constant of about ≤1 μM, e.g., about ≤100 nM, about ≤10 nM, or about ≤1 nM, as measured using standard binding assays, for example, a Biacore-based binding assay.

Antibody molecules of the present invention may be characterized relative to a reference anti-LIV-1 antibody, for example, BR2-22a. Antibody BR2-22a is described in U.S. Pat. No. 8,591,863 and is commercially available from American Type Culture Collection.

### Antibody-Drug Conjugates

In certain embodiments, the anti-LIV1 antibodies of the invention can be combined with antibody drug conjugates (ADCs). An exemplary anti-LIV1-ADC antibody is SGN-LIV1A. Particular ADCs may comprise cytotoxic agents (e.g., chemotherapeutic agents), prodrug converting enzymes, radioactive isotopes or compounds, or toxins (these moieties being collectively referred to as a therapeutic agent). For example, an ADC can be conjugated to a cytotoxic agent such as a chemotherapeutic agent, or a toxin (e.g., a cytostatic or cytocidal agent such as, for example, abrin, ricin A, pseudomonas exotoxin, or diphtheria toxin). Examples of useful classes of cytotoxic agents include, for example, DNA minor groove binders, DNA alkylating agents, and tubulin inhibitors. Exemplary cytotoxic agents include, for example, auristatins, camptothecins, calicheamicins, duocarmycins, etoposides, maytansinoids (e.g., DM1, DM2, DM3, DM4), taxanes, benzodiazepines (e.g., pyrrolo[1,4]benzodiazepines, indolinobenzodiazepines, and oxazolidinobenzodiazepines including pyrrolo[1,4]benzodiazepine dimers, indolinobenzodiazepine dimers, and oxazolidinobenzodiazepine dimers) and vinca alkaloids.

An ADC can be conjugated to a pro-drug converting enzyme. The pro-drug converting enzyme can be recombinantly fused to the antibody or chemically conjugated thereto using known methods. Exemplary pro-drug converting enzymes are carboxypeptidase G2, beta-glucuronidase, penicillin-V-amidase, penicillin-G-amidase, β-lactamase, β-glucosidase, nitroreductase and carboxypeptidase A.

Techniques for conjugating therapeutic agents to proteins, and in particular to antibodies, are well-known. (See, e.g., Alley et al., Current Opinion in Chemical Biology 2010 14: 1-9; Senter, Cancer J., 2008, 14(3): 154-169.) The therapeutic agent can be conjugated in a manner that reduces its activity unless it is cleaved off the antibody (e.g., by hydrolysis, by proteolytic degradation, or by a cleaving agent). In some aspects, the therapeutic agent is attached to the antibody with a cleavable linker that is sensitive to cleavage in the intracellular environment of the LIV-1-expressing cancer cell but is not substantially sensitive to the extracellular environment, such that the conjugate is cleaved from the antibody when it is internalized by the LIV-1-expressing cancer cell (e.g., in the endosomal or, for example by virtue of pH sensitivity or protease sensitivity, in the lysosomal environment or in the caveolear environment). In some embodiments, the therapeutic agent can also be attached to the antibody with a non-cleavable linker.

In certain exemplary embodiments, an ADC can include a linker region between a cytotoxic or cytostatic agent and the antibody. As noted supra, typically, the linker can be cleavable under intracellular conditions, such that cleavage of the linker releases the therapeutic agent from the antibody in the intracellular environment (e.g., within a lysosome or endosome or caveolea). The linker can be, e.g., a peptidyl linker that is cleaved by an intracellular peptidase or protease enzyme, including a lysosomal or endosomal protease. Cleaving agents can include cathepsins B and D and plasmin (see, e.g., Dubowchik and Walker, Pharm. Therapeutics 83:67-123, 1999). Most typical are peptidyl linkers that are cleavable by enzymes that are present in LIV-1-expressing cells. For example, a peptidyl linker that is cleavable by the thiol-dependent protease cathepsin-B, which is highly expressed in cancerous tissue, can be used (e.g., a linker comprising a Phe-Leu or a Val-Cit peptide).

A cleavable linker can be pH-sensitive, i.e., sensitive to hydrolysis, at certain pH values. Typically, a pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, Pharm. Therapeutics 83:67-123, 1999; Neville et al, Biol. Chem. 264: 14653-14661, 1989.) Such linkers are relatively stable under neutral pH conditions, such as those in the blood, but are unstable at below pH 5.5 or 5.0, the approximate pH of the lysosome.

Other linkers are cleavable under reducing conditions (e.g., a disulfide linker). Disulfide linkers include those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT. (See, e.g., Thorpe et al., Cancer Res. 47:5924-5931, 1987; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

A linker can also be a malonate linker (Johnson et al., Anticancer Res. 15: 1387-93, 1995), a maleimidobenzoyl linker (Lau et al., Bioorg-Med-Chem. 3: 1299-1304, 1995), or a 3′-N-amide analog (Lau et al., Bioorg-Med-Chem. 3: 1305-12, 1995).

A linker also can be a non-cleavable linker, such as an maleimido-alkylene or maleimide-aryl linker that is directly attached to the therapeutic agent and released by proteolytic degradation of the antibody.

Typically, a linker is not substantially sensitive to the extracellular environment, meaning that no more than about 20%, typically no more than about 15%, more typically no more than about 10%, and even more typically no more than about 5%, no more than about 3%, or no more than about 1% of the linkers in a sample of the ADC are cleaved when the ADC is present in an extracellular environment (e.g., in plasma). Whether a linker is not substantially sensitive to the extracellular environment can be determined, for example, by incubating independently with plasma both (a) the ADC (the “ADC sample”) and (b) an equal molar amount of unconjugated antibody or therapeutic agent (the “control sample”) for a predetermined time period (e.g., 2, 4, 8, 16, or 24 hours) and then comparing the amount of unconjugated antibody or therapeutic agent present in the ADC sample with that present in control sample, as measured, for example, by high performance liquid chromatography.

A linker can also promote cellular internalization, e.g., when conjugated to the therapeutic agent (i.e., in the milieu of the linker-therapeutic agent moiety of the ADC or ADC derivate as described herein). Alternatively, the linker can promote cellular internalization when conjugated to both the therapeutic agent and the antibody (i.e., in the milieu of the ADC as described herein).

An anti-LIV-1 antibody can be conjugated to a linker via a heteroatom of the antibody. These heteroatoms can be present on the antibody in its natural state or can be introduced into the anti-LIV-1 antibody. In some aspects, the anti-LIV-1 antibody will be conjugated to the linker via a sulfur atom of a cysteine residue. Methods of conjugating linker and drug-linkers to antibodies are known in the art.

Exemplary antibody-drug conjugates include auristatin based antibody-drug conjugates meaning that the drug component is an auristatin drug. Auristatins bind tubulin, have been shown to interfere with microtubule dynamics and nuclear and cellular division, and have anticancer activity. Typically the auristatin based antibody-drug conjugate comprises a linker between the auristatin drug and the anti-LIV-1 antibody. The linker can be, for example, a cleavable linker (e.g., a peptidyl linker) or a non-cleavable linker (e.g., linker released by degradation of the antibody). Auristatins include MMAF and MMAE. The synthesis and structure of exemplary auristatins are described in U.S. Pat. Nos. 7,659,241, 7,498,298, 7,968,687, and U.S. Pub. Nos. 2009/0111756 and 2009/0018086, each of which is incorporated herein by reference in its entirety and for all purposes.

In certain embodiments, an anti-LIV1 antibody or antigen-binding fragment thereof can be combined with an antibody drug conjugate (ADC) and may have a ratio of drug moieties per antibody of about 1 to about 8. In certain embodiments, an anti-LIV1 antibody or antigen-binding fragment thereof can be combined with an ADC and may have a ratio of drug moieties per antibody of about 2 to about 5. In some embodiments, the ratio of drug moieties per antibody is 1, 2, 3, 4, 5, 6, 7, 8, 9, or 10. In certain exemplary embodiments, an anti-LIV1 antibody or antigen-binding fragment thereof can be combined with an ADC and have a ratio of drug moieties per antibody of 4. Methods of determining the ratio of drug moieties per antibody or antigen-binding fragment thereof of an ADC are readily known to those skilled in the art.

### III. Therapeutic Applications

The invention provides methods of treating disorders associated with cells that express LIV-1, e.g., cancers (e.g., breast cancers such as locally advanced breast cancer or metastatic breast cancer). As a result, the invention provides a method of treating a subject, for example, a subject with breast cancer, using the anti-LIV1 antibodies and antigen-binding fragments thereof (e.g., a LIV1-ADC) described herein. The method comprises administering an effective amount of an anti-LIV1 antibody or a composition comprising an anti-LIV1 antibody or an antigen-binding fragment thereof (e.g., a LIV1-ADC) to a subject in need thereof.

As used herein, the terms “subject” and “patient” refer to organisms to be treated by the methods of the present invention. Such organisms preferably include, but are not limited to, mammals (e.g., murines, simians, equines, bovines, porcines, canines, felines, and the like), and more preferably includes humans. As used herein, the terms “treat,” “treatment” and “treating” include any effect, e.g., lessening, reducing, modulating, ameliorating or eliminating, that results in the improvement of the condition, disease, disorder, and the like, or ameliorating a symptom thereof, such as for example, reduced number of cancer cells, reduced tumor size, reduced rate of cancer cell infiltration into peripheral organs, or reduced rate of tumor metastasis or tumor growth.

Positive therapeutic effects in cancer can be measured in a number of ways (See, W. A. Weber, J. Null. Med. 50:1S-10S (2009); Eisenhauer et al., supra). In certain exemplary embodiments, response to an anti-LIV1 antibody or an antigen-binding fragment thereof (e.g., a LIV1-ADC) is assessed using RECIST 1.1 criteria. In some embodiments, the treatment achieved by a therapeutically effective amount is any of a partial response (PR), a complete response (CR), progression free survival (PFS), disease free survival (DFS), objective response (OR) or overall survival (OS). The dosage regimen of a therapy described herein that is effective to treat breast cancer patient may vary according to factors such as the disease state, age, and weight of the patient, and the ability of the therapy to elicit an anti-cancer response in the subject. While an embodiment of the treatment method, medicaments and uses of the present invention may not be effective in achieving a positive therapeutic effect in every subject, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“RECIST 1.1 Response Criteria” as used herein means the definitions set forth in Eisenhauer et al., E. A. et al., Eur. J Cancer 45:228-247 (2009) for target lesions or non-target lesions, as appropriate, based on the context in which response is being measured.

“Tumor” as it applies to a subject diagnosed with, or suspected of having, cancer (e.g., breast cancer), refers to a malignant or potentially malignant neoplasm or tissue mass of any size. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load,” refers to the total amount of tumor material distributed throughout the body. Tumor burden refers to the total number of cancer cells or the total size of tumor(s) throughout the body, including lymph nodes and bone narrow. Tumor burden can be determined by a variety of methods known in the art, such as, e.g., by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

As used herein, the term “effective amount” refers to the amount of a compound (e.g., an anti-LIV1 antibody or antigen-binding fragment thereof) sufficient to effect beneficial or desired results. An effective amount of an anti-LIV1 antibody or antigen-binding fragment thereof (e.g., a LIV1-ADC) can be administered in one or more administrations, applications or dosages and is not intended to be limited to a particular formulation or administration route. Generally, a therapeutically effective amount of an anti-LIV1 antibody or antigen-binding fragment thereof (e.g., a LIV1-ADC) is in the range of 0.5 mg/kg to 2.8 mg/kg at a maximum dose of about 200 mg. The dosage administered can vary depending upon known factors, such as the pharmacodynamic characteristics of the particular agent, and its mode and route of administration; the age, health, and weight of the recipient; the type and extent of disease or indication to be treated, the nature and extent of symptoms, kind of concurrent treatment, frequency of treatment, and the effect desired. The initial dosage can be increased beyond the upper level in order to rapidly achieve the desired blood-level or tissue-level. Alternatively, the initial dosage can be smaller than the optimum, and the daily dosage may be progressively increased during the course of treatment. Dosing frequency can vary, depending on factors such as route of administration, dosage amount, serum half-life of the antibody, and the disease being treated. Exemplary dosing frequencies are once per day, once per week, once every two weeks and once every three weeks. Formulation of monoclonal antibody-based drugs is within ordinary skill in the art. In some embodiments, a monoclonal antibody is lyophilized, and then reconstituted in buffered saline, at the time of administration.

In some embodiments, an anti-LIV1 antibody or antigen-binding fragment thereof (e.g., a LIV1-ADC) is administered to a patient who failed to achieve a sustained response after prior therapy (e.g., after failed or ineffective therapy with a systemic anti-cancer therapy that is not an anti-LIV1 antibody or antigen-binding fragment thereof (e.g., a LIV1-ADC)), i.e., is cancer treatment-experienced.

In some embodiments, a medicament comprising an anti-LIV1 antibody or antigen-binding fragment thereof (e.g., a LIV1-ADC), as described above, may be provided as a liquid formulation or prepared by reconstituting a lyophilized powder with sterile water for injection prior to use.

In certain embodiments, the dosing regimen will comprise administering an anti-LIV1 antibody or antigen-binding fragment thereof (e.g., a LIV1-ADC) at a dose of about 2.5 mg/kg of a subject's body weight at intervals of about 21 days (±2 days) throughout the course of treatment. In certain embodiments, an anti-LIV1 antibody or antigen-binding fragment thereof (e.g., a LIV1-ADC) is used at a dose of less than about 200 mg every 3 weeks.

In certain embodiments, the dosing regimen will comprise administering an anti-LIV1 antibody or antigen-binding fragment thereof (e.g., a LIV1-ADC) at a dose of about 2.5 mg/kg of a subject's body weight at intervals of about 21 days (±2 days) throughout the course of treatment. In certain embodiments, an anti-LIV1 antibody or antigen-binding fragment thereof (e.g., a LIV1-ADC) is used at a dose of less than or equal to about 250 mg every 3 weeks. In certain embodiments, an anti-LIV1 antibody or antigen-binding fragment thereof (e.g., a LIV1-ADC) is used at a dose of less than or equal to 250 mg every 3 weeks. In certain embodiments, the subject is further administered granulocyte colony stimulating factor (GCSF). In certain embodiments, if the anti-LIV1 antibody or antigen-binding fragment thereof (e.g., a LIV1-ADC) is used at a dose of greater than or equal to about 200 mg and less than or equal to about 250 mg every 3 weeks, the subject is further administered GCSF. In certain embodiments, if the anti-LIV1 antibody or antigen-binding fragment thereof (e.g., a LIV1-ADC) is used at a dose of greater than or equal to 200 mg and less than or equal to 250 mg every 3 weeks, the subject is further administered GCSF. In certain embodiments, the GCSF is administered prophylactically. In certain embodiments, the GCSF is recombinant human GCSF. In certain embodiments, the GCSF is filgrastim (NEUPOGEN®). In certain embodiments, the GCSF is PEG-filgrastim (NEULASTA®) In certain embodiments, the GCSF is lenograstim (GRANOCYTE®). In certain embodiments, the GCSF is tbo-filgrastim (GRANIX®).

In certain embodiments, a subject will be administered a parenteral dosing, e.g., an intravenous (IV) infusion, of a medicament comprising an anti-LIV1 antibody or antigen-binding fragment thereof (e.g., a LIV1-ADC).

In a particular embodiment of the invention, an anti-LIV1 antibody or antigen-binding fragment thereof (e.g., a LIV1-ADC) is administered to a subject in a liquid medicament at a dose selected from the group consisting of about 0.5 mg/kg of body weight every three weeks (Q3W) or every 21 days (Q21D), about 1.0 mg/kg of body weight Q3W or Q21D, about 1.5 mg/kg of body weight Q3W or Q21D, about 2.0 mg/kg of body weight Q3W or Q21D, about 2.5 mg/kg of body weight Q3W or Q21D, or about 2.8 mg/kg of body weight Q3W or Q21D, and maximum equivalents of any of these doses, such as, e.g., less than about 200 mg Q3W or Q21D.

In a particular embodiment of the invention, an anti-LIV1 antibody or antigen-binding fragment thereof (e.g., a LIV1-ADC) is administered to a subject in a liquid medicament at a dose selected from the group consisting of about 0.5 mg/kg of body weight every three weeks (Q3W) or every 21 days (Q21D), about 1.0 mg/kg of body weight Q3W or Q21D, about 1.5 mg/kg of body weight Q3W or Q21D, about 2.0 mg/kg of body weight Q3W or Q21D, about 2.5 mg/kg of body weight Q3W or Q21D, or about 2.8 mg/kg of body weight Q3W or Q21D, and maximum equivalents of any of these doses, such as, e.g., less than or equal to about 250 mg Q3W or Q21D. In certain embodiments, the subject is further administered GCSF. In certain embodiments, if the dose is greater than or equal to about 200 mg and less than or equal to about 250 mg Q3W or Q21D, the subject is further administered GCSF. In certain embodiments, the subject is further administered GCSF. In certain embodiments, if the dose is greater than or equal to 200 mg and less than or equal to 250 mg Q3W or Q21D, the subject is further administered GCSF. In certain embodiments, the GCSF is administered prophylactically. In certain embodiments, the GCSF is recombinant human GCSF. In certain embodiments, the GCSF is filgrastim (NEUPOGEN®). In certain embodiments, the GCSF is PEG-filgrastim (NEULASTA®) In certain embodiments, the GCSF is lenograstim (GRANOCYTE®). In certain embodiments, the GCSF is tbo-filgrastim (GRANIX®).

In some embodiments, an anti-LIV1 antibody or antigen-binding fragment thereof (e.g., a LIV1-ADC) is provided in a dosage of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 191 mg, about 192 mg, about 193 mg, about 194 mg, about 195 mg, about 196 mg, about 197 mg, about 198 mg, about 199 mg or about 200 mg. In certain exemplary embodiments, an anti-LIV1 antibody or antigen-binding fragment thereof (e.g., a LIV1-ADC) is provided in a dosage of less than about 200 mg, e.g., at a dosage of about 200 mg, at a dosage of about 199 mg, about 198 mg, about 197 mg, about 196 mg, about 195 mg, about 190 mg, about 185 mg, about 180 mg, about 175 mg, about 170 mg, about 165 mg, about 160 mg, about 155 mg, about 150 mg, about 145 mg, about 140 mg, about 135 mg, about 130 mg, about 125 mg, about 120 mg, about 115 mg, about 110 mg, about 105 mg, or about 100 mg.

In some embodiments, an anti-LIV1 antibody or antigen-binding fragment thereof (e.g., a LIV1-ADC) is provided in a dosage of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 245 mg or about 250 mg. In certain exemplary embodiments, an anti-LIV1 antibody or antigen-binding fragment thereof (e.g., a LIV1-ADC) is provided in a dosage of less than or equal to about 250 mg, e.g., at a dosage of about 250 mg, at a dosage of about 245 mg, about 240 mg, about 235 mg, about 230 mg, about 225 mg, about 220 mg, about 215 mg, about 210 mg, about 205 mg, about 200 mg, about 195 mg, about 190 mg, about 185 mg, about 180 mg, about 175 mg, about 170 mg, about 165 mg, about 160 mg, about 155 mg, about 150 mg, about 145 mg, about 140 mg, about 135 mg, about 130 mg, about 125 mg, about 120 mg, about 115 mg, about 110 mg, about 105 mg, or about 100 mg.

In certain exemplary embodiments, the present invention provides a method for treating cancer in a cell, tissue, organ, animal or patient. In particular embodiments, the present invention provides a method for treating a breast cancer in a human.

Certain breast cancers show detectable levels of LIV-1 measured at either the protein (e.g., by immunoassay using one of the exemplified antibodies) or the mRNA level. In certain embodiments, a breast cancer shows elevated levels of LIV-1 relative to non-cancerous tissue or cells of the same type, e.g., other breast cells or breast tissues from the same patient. In other embodiments, a breast cancer shows similar levels of LIV-1 relative to non-cancerous breast tissue or breast cells of the same type, e.g., from the same patient.

An exemplary level of LIV-1 protein on breast cancer cells amenable to treatment is 5,000-150,000 LIV-1 proteins per cell, although breast cancers associated with higher or lower levels can be treated. Optionally, LIV-1 levels (e.g., LIV-1 protein levels) in a breast cancer from a subject are measured before performing treatment.

Exemplary breast cancers are those that express LIV-1 in a cell expressing the cancer (i.e., LIV1-expressing cancers). In certain exemplary embodiments, a breast cancer is selected from the group consisting of carcinomas, sarcomas, phyllodes, Paget disease, and angiosarcomas. The breast cancer may be in situ (e.g., ductal carcinoma in situ (DCIS), lobular carcinoma in situ (LCIS) and the like) or invasive/infiltrating (e.g., invasive ductal carcinoma (IDC), invasive lobular carcinoma (ILC), inflammatory breast cancer (IBC) and the like).

Breast cancer may have the following characteristics: estrogen receptor positive (ER+); progesterone receptor positive (PR+); hormone receptor negative (HR−); HER2 gene overexpressing (HER2+); HER2 gene wild-type or under-expressing (HER2−); group 1 (luminal A), i.e., ER+/PR+/HER2−; group 2 (luminal B), i.e., ER+/PR−/HER2+; group 3 (HER2+), i.e., ER−/PR−/HER2+; and group 4 (basal-like or triple negative (TN)), i.e., ER−/PR−/HER2−.

A breast cancer can further be categorized as grade 1, 2 or 3. Grade 1 or well-differentiated (score 3, 4, or 5) breast cancer comprises cells that are slower-growing, and look more like normal breast tissue than the higher grades of breast cancer. Grade 2 or moderately differentiated (score 6, 7) breast cancer has cells that grow at a speed of and look like cells somewhere between grades 1 and 3. Grade 3 or poorly differentiated (score 8, 9) breast cancer has cells that look very different from normal cells and typically grow and spread faster than grades 1 or 2.

In certain exemplary embodiments, a breast cancer is an incurable, unresectable, locally advanced or metastatic breast cancer (LA/MBC). In certain embodiments, a breast cancer is either a triple negative (TN) (ER−/PR−/HER2−) breast cancer, an ER− and/or PR+/HER2− breast cancer, and an LA/MBC breast cancer. In certain exemplary embodiments, the breast cancer is HER2+ and LA/MBC. In certain exemplary embodiments, a breast cancer is TN and LA/MBC. In certain exemplary embodiments, a breast cancer is selected from the group consisting of a TN breast cancer, a metastatic breast cancer, and a metastatic, TN breast cancer.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing and method steps.

### IV. Pharmaceutical Compositions and Formulations

For therapeutic use, an anti-LIV1 antibody or antigen-binding fragment thereof (e.g., a LIV1-ADC) is combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Accordingly, anti-LIV1 antibody or antigen-binding fragment thereof (e.g., a LIV1-ADC) compositions of the present invention can comprise at least one of any suitable excipients, such as, but not limited to, diluent, binder, stabilizer, buffers, salts, lipophilic solvents, preservative, adjuvant or the like. Pharmaceutically acceptable excipients are preferred. Non-limiting examples of, and methods of preparing such sterile solutions are well known in the art, such as, but not limited to, those described in Gennaro, Ed., Remington's Pharmaceutical Sciences, 18th Edition, Mack Publishing Co. (Easton, Pa.) 1990. Pharmaceutically acceptable carriers can be routinely selected that are suitable for the mode of administration, solubility and/or stability of the antibody molecule, fragment or variant composition as well known in the art or as described herein.

Suitable pharmaceutical excipients and/or additives for use in the antibody molecule compositions according to the invention are known in the art, e.g., as listed in “Remington: The Science & Practice of Pharmacy,” 19th ed., Williams & Williams, (1995), and in the “Physician's Desk Reference,” 52nd ed., Medical Economics, Montvale, N.J. (1998).

Pharmaceutical compositions containing an anti-LIV1 antibody or antigen-binding fragment thereof (e.g., a LIV1-ADC) as disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, and rectal administration. A preferred route of administration for monoclonal antibodies is IV infusion. Useful formulations can be prepared by methods known in the pharmaceutical art. For example, see Remington's Pharmaceutical Sciences (1990) supra. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

Pharmaceutical formulations are preferably sterile. Sterilization can be accomplished by any suitable method, e.g., filtration through sterile filtration membranes. Where the composition is lyophilized, filter sterilization can be conducted prior to or following lyophilization and reconstitution.

The compositions of this invention may be in a variety of forms. These include, for example, liquid, semi-solid and solid dosage forms, such as liquid solutions (e.g., injectable and infusible solutions), dispersions or suspensions, and liposomes. The particular form depends on the intended mode of administration and therapeutic application. In exemplary embodiments, compositions provided are in the form of injectable or infusible solutions. Exemplary administration is parenteral (e.g., intravenous, subcutaneous, intraocular, intraperitoneal, intramuscular). In an exemplary embodiment, the preparation is administered by intravenous infusion or injection. In another preferred embodiment, the preparation is administered by intramuscular or subcutaneous injection.

The phrases “parenteral administration” and “administered parenterally” as used herein means modes of administration other than enteral and topical administration, usually by injection, and includes, without limitation, intravenous, intramuscular, subcutaneous, intraarterial, intrathecal, intracapsular, intraorbital, intravitreous, intracardiac, intradermal, intraperitoneal, transtracheal, inhaled, subcutaneous, subcuticular, intraarticular, subcapsular, subarachnoid, intraspinal, epidural and intrasternal injection and infusion.

Exemplary dosages of an anti-LIV1 antibody or antigen-binding fragment thereof (e.g., a LIV1-ADC) are about 0.5 mg/kg of a subject's body weight, about 1.0 mg/kg of a subject's body weight, about 1.5 mg/kg of a subject's body weight, about 2.0 mg/kg of a subject's body weight, about 2.5 mg/kg of a subject's body weight, or about 2.8 mg/kg of a subject's body weight. In a particular embodiment, an exemplary dose of an anti-LIV1 antibody or antigen-binding fragment thereof (e.g., a LIV1-ADC) is about 2.5 mg/kg of a subject's body weight. In another particular embodiment, a maximum exemplary dose of an anti-LIV1 antibody or antigen-binding fragment thereof (e.g., a LIV1-ADC) is about 200 mg per cycle. In another particular embodiment, a maximum exemplary dose of an anti-LIV1 antibody or antigen-binding fragment thereof (e.g., a LIV1-ADC) is about 250 mg per cycle.

In certain exemplary embodiments, a subject is administered a dose of about 2.5 mg/kg, at a maximum dose of about 200 mg, once every three weeks. In certain exemplary embodiments, a subject is administered an intravenous dose of about 2.5 mg/kg, at a maximum dose of about 200 mg, once every three weeks.

In certain exemplary embodiments, a subject is administered a dose of about 2.5 mg/kg, at a maximum dose of about 250 mg, once every three weeks. In certain exemplary embodiments, a subject is administered an intravenous dose of about 2.5 mg/kg, at a maximum dose of about 250 mg, once every three weeks. In certain exemplary embodiments, the subject is further administered GCSF. In certain exemplary embodiments, if the anti-LIV1 antibody or antigen-binding fragment thereof (e.g., a LIV1-ADC) is used at a dose of greater than or equal to about 200 mg and less than or equal to about 250 mg once every three weeks, the subject is further administered GCSF. In certain exemplary embodiments, if the anti-LIV1 antibody or antigen-binding fragment thereof (e.g., a LIV1-ADC) is used at a dose of greater than or equal to 200 mg and less than or equal to 250 mg once every three weeks, the subject is further administered GCSF. In certain embodiments, the GCSF is administered prophylactically. In certain embodiments, the GCSF is recombinant human GCSF. In certain embodiments, the GCSF is filgrastim (NEUPOGEN®). In certain embodiments, the GCSF is PEG-filgrastim (NEULASTA®). In certain embodiments, the GCSF is lenograstim (GRANOCYTE®). In certain embodiments, the GCSF is tbo-filgrastim (GRANIX®).

The present invention provides a kit, comprising packaging material and at least one vial comprising a solution of at least one an anti-LIV1 antibody or antigen-binding fragment thereof (e.g., a LIV1-ADC) with the prescribed buffers and/or preservatives, optionally in an aqueous diluent. The concentration of preservative used in the formulation is a concentration sufficient to yield an anti-microbial effect. Such concentrations are dependent on the preservative selected and are readily determined by the skilled artisan.

Various delivery systems can be used to administer anti-LIV1 antibodies or antigen-binding fragments thereof to a subject. In certain exemplary embodiments, administration of an anti-LIV1 antibody or antigen-binding fragment thereof (e.g., a LIV1-ADC) is by intravenous infusion.

Any of the formulations described above can be stored in a liquid or frozen form and can be optionally subjected to a preservation process. In some embodiments, the formulations described above are lyophilized, i.e., they are subjected to lyophilization. In some embodiments, the formulations described above are subjected to a preservation process, for example, lyophilization, and are subsequently reconstituted with a suitable liquid, for example, water. By lyophilized, it is meant that the composition has been freeze-dried under a vacuum. Lyophilization typically is accomplished by freezing a particular formulation such that the solutes are separated from the solvent(s). The solvent is then removed by sublimation (i.e., primary drying) and next by desorption (i.e., secondary drying).

The formulations of the present invention can be used with the methods described herein or with other methods for treating disease. The anti-LIV1 antibody or antigen-binding fragment thereof (e.g., LIV1-ADC) formulations may be further diluted before administration to a subject. In some embodiments, the formulations will be diluted with saline and held in IV bags or syringes before administration to a subject. Accordingly, in some embodiments, the methods for treating a LIV-1-expressing cancer in a subject will comprise administering to a subject in need thereof a weekly dose of a pharmaceutical composition comprising an anti-LIV1 antibody or antigen-binding fragment thereof (e.g., a LIV1-ADC).

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting. All patents, patent applications and references described herein are incorporated by reference in their entireties for all purposes.

### EXAMPLES

### Example 1: Phase 1 Study of the Antibody-Drug Conjugate SGN-LIV1A in Patients with Heavily Pretreated Triple-Negative Metastatic Breast Cancer

### Methods

This ongoing, phase 1 study evaluated safety, tolerability, pharmacokinetics, and anti-tumor activity of SGN-LIV1A (q3wks IV) in women with LIV-1-positive, unresectable, locally advanced or metastatic breast cancer (LA/MBC) (NCT01969643). Patients with measurable disease and ≥2 prior cytotoxic regimens for LA/MBC were eligible. Patients with ≥Grade 2 neuropathy were excluded. Response was assessed per RECIST v1.1; pts with stable disease (SD) or better could continue treatment until disease progression or intolerable toxicity. At completion of dose escalation in hormone receptor-positive/HER2-negative (HR+/HER2−) and triple-negative (TN) patients, expansion cohorts were opened to further evaluate safety and antitumor activity of monotherapy in TN patients. Tumor biopsies were evaluated for LIV1 expression.

### Results

To date, 69 patients (18 HR+/HER2−, 51 TN) have received a median of 3 cycles (range, 1-12) of SGN-LIV1A at doses of 0.5 to 2.8 mg/kg. Median patient age was 56 years. Patients had a median of three prior cytotoxic regimens for LA/MBC. 58 patients had visceral disease and 37 patients had bone metastases. No dose-limiting toxicities (DLTs) occurred in 19 DLT-evaluable patients. The maximum tolerated dose was not exceeded at 2.8 mg/kg. Expansion cohorts of TN pts were opened at 2.0 and 2.5 mg/kg.

Treatment-emergent adverse events (AEs) reported in ≥25% of patients were fatigue (59%), nausea (51%), peripheral neuropathy (44%), alopecia (36%), decreased appetite (33%), constipation (30%), abdominal pain (25%), diarrhea (25%), and neutropenia (25%). Most AEs were Grade 1/2. AEs ≥Grade 3 included neutropenia (25%) and anemia (15%). Febrile neutropenia occurred in 2 patients whose total dose exceeded 200 mg per cycle, including one treatment-related death due to sepsis. No other treatment-related deaths occurred on-study. Seven patients discontinued treatment due to AEs.

In dose escalation, activity was observed in 17 efficacy evaluable (EE) HR+/HER2− pts, with a disease control rate (DCR=CR+PR+SD) of 59% (10 SD), including one patient with SD ≥24 wks. Among the 44 EE TN patients (dose escalation plus expansion cohorts), the objective response rate (ORR) was 32% (14 PR) with a confirmed PR rate of 21%, DCR was 64% (14 PR, 14 SD), and clinical benefit rate (CBR=CR+PR+SD ≥24 weeks) was 36% (16 patients). For TN patients, median PFS was 11.3 weeks (95% CI: 6.1, 17.1). 10 patients remain on treatment.

Of 631 MBC tumor samples of all clinical subtypes evaluated for LIV-1, 91% were positive, and 75% had moderate-to-high expression (H-score ≥100).

At the completion of dose escalation, multiple expansion cohorts for SGN-LIV1A monotherapy (Part A) treatment were opened to enroll up to 15 patients each with specific breast cancer subtypes at a recommended dose level to further define the safety and antitumor activity. Following an analysis of safety and activity in the 2.0 mg/kg versus 2.5 mg/kg dose cohorts, the recommended phase 2 dose was determined to be 2.5 mg/kg (maximum dose of 200 mg per cycle)

A review of the safety data available to date found that the incidence of Grade 3 or higher neutropenia AEs in the 2.5 mg/kg dose level of Part A (57%) was higher than that in the overall monotherapy study population (39%). Additionally, one case of neutropenia in the 2.5 mg/kg group resulted in death due to sepsis. As a result, a decision was made to evaluate the 2 mg/kg dose level in expansion cohorts. Patients enrolled on or after this decision date received a starting dose of 2 mg/kg, and patients who had previously received 2.5 mg/kg of SGN-LIV1A in earlier cycles had their dose reduced to 2 mg/kg for subsequent doses.

Enrollment in the mTNBC 2.0 mg/kg expansion cohort is nearing completion with 10 patients remaining on treatment at the time of the data cut. A comparison of safety between 2.0 mg/kg (N=26) and 2.5 mg/kg (doses ≤200 mg) (N=18) revealed no febrile neutropenic events or neutropenia-associated SAEs. In contrast, at doses of 2.5 mg/kg >200 mg (N=11) neutropenia was more common, with 5 of 11 patients experiencing SAEs associated with neutropenia. Febrile neutropenia occurred in 2 of 11 patients, including the one treatment related death due to sepsis. No other treatment related deaths occurred on study.

### Example 2: Phase 1 Study of the Antibody-Drug Conjugate SGN-LIV1A in Patients with Heavily Pretreated Triple-Negative Metastatic Breast Cancer

### Methods

This study is a continuation of the dose expansion cohort study for SGN-LIV1A monotherapy (Part A) of the phase 1 study described in Example 1, which includes data from 22 additional administrations of SGN-LIV1A (q3wks IV). Patients were as described in Example 1. These additional administrations were conducted to assess the safety of a total maximum dose of 250 mg per cycle. Patients were dosed at 2.5 mg/kg and each of these 22 additional administrations were greater than or equal to 200 mg per cycle due to the patients having a weight greater than or equal to 80 kg.

### Results

Of the 22 additional administrations of greater than or equal to 200 mg up to the maximum dose of 250 mg per cycle, 7 administrations of SGN-LIV1A were co-administered with granulocyte colony stimulating factor (GCSF) and 15 administrations of SGN-LIV1A were not. Of the 15 administrations of SGN-LIV1A that were not co-administered with GCSF, 5 resulted in the development of neutropenia (33.3%). However, none of the 7 administrations of SGN-LIV1A that were co-administered with GCSF resulted in the development of neutropenia. These results indicate that the incidence of neutropenia in patients receiving dosages greater than or equal to 200 mg up to the maximum dose of 250 mg per cycle can be significantly reduced through the use of GCSF.

What is claimed:

1. A method of treating a subject having or at risk of having a LIV-1-associated cancer, comprising:
administering to the subject a therapeutically effective dose of an antibody or an antigen-binding fragment thereof that specifically binds human LIV-1,
wherein the dose administered is less than about 200 mg of the antibody or antigen-binding fragment thereof per treatment cycle, and
wherein the antibody or antigen-binding fragment thereof comprises a heavy chain variable region (HCVR) having at least 95% identity to SEQ ID NO: 1, and a light chain variable region (LCVR) having at least 95% identity to SEQ ID NO: 2.

2. A method of treating a subject having or at risk of having a LIV-1-associated cancer, comprising:
administering to the subject a therapeutically effective dose of an antibody or an antigen-binding fragment thereof that specifically binds human LIV-1,
wherein the dose administered is less than or equal to about 250 mg of the antibody or antigen-binding fragment thereof per treatment cycle,
wherein the antibody or antigen-binding fragment thereof comprises a heavy chain variable region (HCVR) having at least 95% identity to SEQ ID NO: 1, and a light chain variable region (LCVR) having at least 95% identity to SEQ ID NO: 2, and
wherein if the dose administered is greater than or equal to about 200 mg of the antibody or antigen-binding fragment thereof per treatment cycle, the method further comprises administering granulocyte colony stimulating factor (GCSF) to the subject.

3. The method of claim 2, wherein, if administered, the GCSF is administered prophylactically.

4. A method of treating a subject having or at risk of having a LIV-1-associated cancer, comprising:
administering to the subject granulocyte colony stimulating factor (GCSF),
administering to the subject a therapeutically effective dose of an antibody or an antigen-binding fragment thereof that specifically binds human LIV-1,
wherein the dose administered is greater than or equal to about 200 mg and less than or equal to about 250 mg of the antibody or antigen-binding fragment thereof per treatment cycle,
wherein the antibody or antigen-binding fragment thereof comprises a heavy chain variable region (HCVR) having at least 95% identity to SEQ ID NO: 1, and a light chain variable region (LCVR) having at least 95% identity to SEQ ID NO: 2.

5. The method of claim 4, wherein the GCSF is administered prophylactically.

6. The method of any one of claims 1-5, wherein the LIV-1-associated cancer is a breast cancer.

7. The method of claim 6, wherein the breast cancer is a triple negative breast cancer.

8. The method of claim 6, wherein the breast cancer is a metastatic breast cancer.

9. The method of claim 6, wherein the breast cancer is a triple-negative, metastatic breast cancer.

10. The method of claim 6, wherein the breast cancer is a hormone receptor-positive, metastatic breast cancer.

11. The method of any one of claims 1-10, wherein the treatment cycle is about every three weeks (Q3W).

12. The method of any one of claims 1-11, wherein the dose is about 2.5 mg/kg of body weight of the subject.

13. The method of any one of claims 1-12, wherein the antibody or antigen-binding fragment thereof is conjugated to monomethyl auristatin E (MMAE):

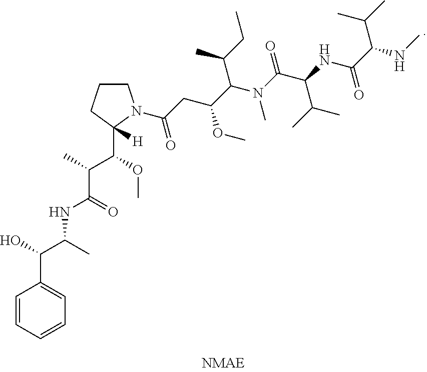 [SMILES: CC[C@H](C)C([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

14. The method of any one of claims 1-13, wherein the antibody or antigen-binding fragment thereof is conjugated to valine-citrulline-monomethyl auristatin E (vcMMAE):

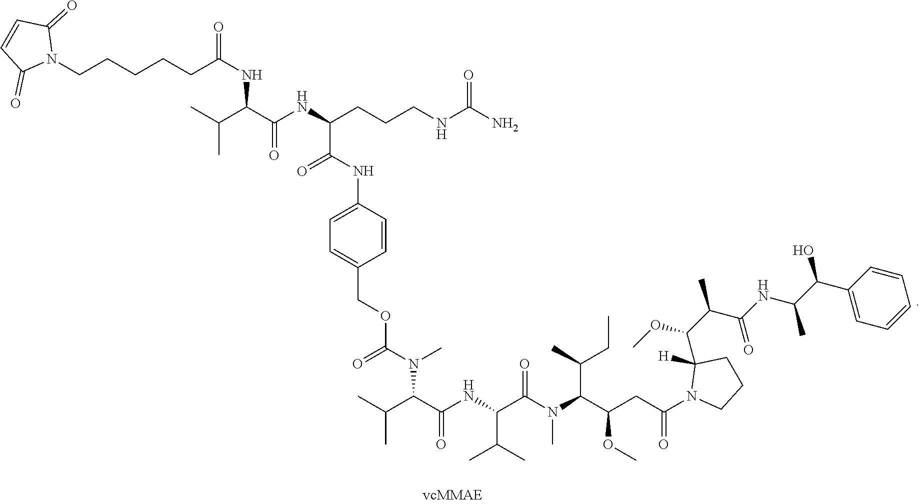 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

15. The method of claim 14, wherein a vcMMAE to antibody or antigen-binding fragment thereof ratio is from about 1 to about 8.

16. The method of claim 15, wherein the vcMMAE to antibody or antigen-binding fragment thereof ratio is about 4.

17. The method of any one of claims 1-16, wherein the HCVR has at least 97% sequence identity to SEQ ID NO: 1 and the LCVR has at least 97% sequence identity to SEQ ID NO: 2.

18. The method of any one of claims 1-17, wherein the HCVR has at least 99% sequence identity to SEQ ID NO: 1 and the LCVR has at least 99% sequence identity to SEQ ID NO: 2.

19. A method of treating a subject having or at risk of having a LIV-1-associated cancer, comprising:
administering to the subject a therapeutically effective dose of an antibody or an antigen-binding fragment thereof that specifically binds human LIV-1,
wherein the dose administered is less than about 200 mg of the antibody or antigen-binding fragment thereof per treatment cycle,
wherein the antibody or antigen-binding fragment thereof comprises an HCVR having at least 95% identity to SEQ ID NO: 1, and an LCVR having at least 95% identity to SEQ ID NO: 2, and
wherein the antibody or antigen-binding fragment thereof is conjugated to vcMMAE:

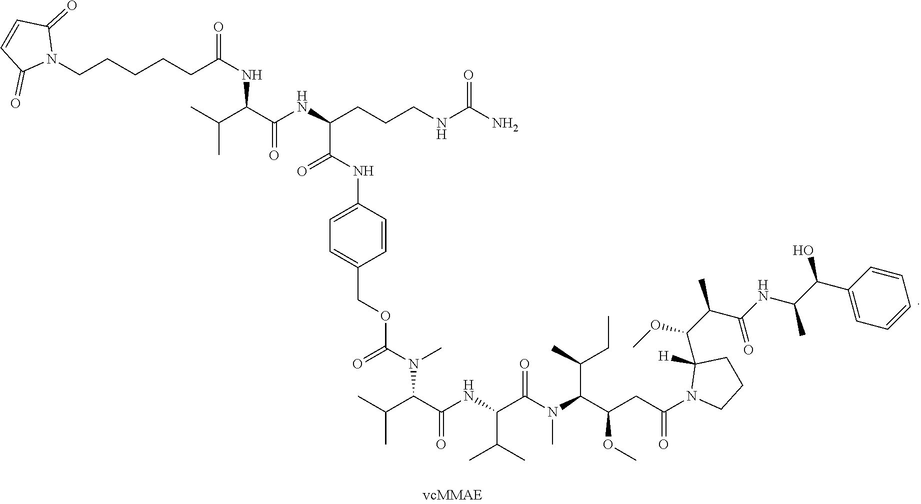 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

20. A method of treating a subject having or at risk of having a LIV-1-associated cancer, comprising:
administering to the subject a therapeutically effective dose of an antibody or an antigen-binding fragment thereof that specifically binds human LIV-1,
wherein the dose administered is less than or equal to about 250 mg of the antibody or antigen-binding fragment thereof per treatment cycle,
wherein the antibody or antigen-binding fragment thereof comprises an HCVR having at least 95% identity to SEQ ID NO: 1, and an LCVR having at least 95% identity to SEQ ID NO: 2,
wherein the antibody or antigen-binding fragment thereof is conjugated to vcMMAE: 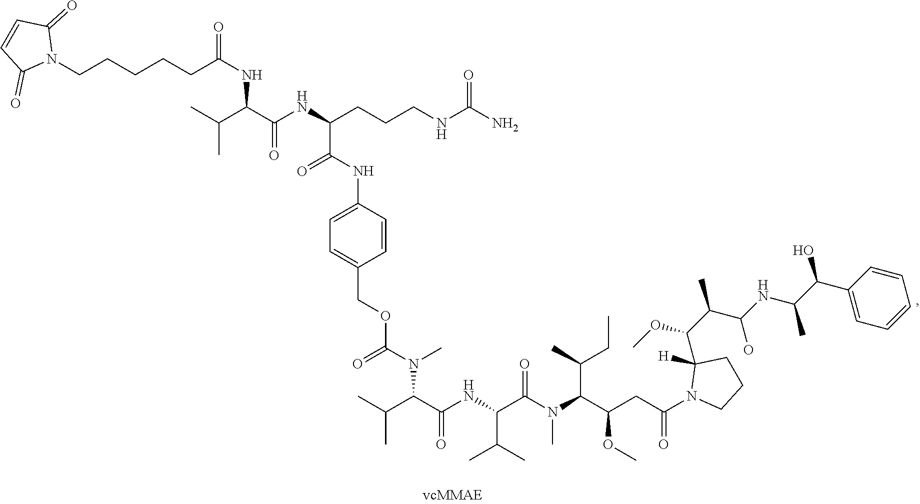 [SMILES: CC[C@H](C)[C@@H]([C@@H](CC(=O)N1CCC[C@H]1[C@H](OC)[C@@H](C)C(O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]
and
wherein if the dose administered is greater than or equal to about 200 mg of the antibody or antigen-binding fragment thereof per treatment cycle, the method further comprises administering granulocyte colony stimulating factor (GCSF) to the subject.

21. The method of claim 20, wherein, if administered, the GCSF is administered prophylactically.

22. A method of treating a subject having or at risk of having a LIV-1-associated cancer, comprising:
administering to the subject granulocyte colony stimulating factor (GCSF),
administering to the subject a therapeutically effective dose of an antibody or an antigen-binding fragment thereof that specifically binds human LIV-1,
wherein the dose administered is greater than or equal to about 200 mg and less than or equal to about 250 mg of the antibody or antigen-binding fragment thereof per treatment cycle,
wherein the antibody or antigen-binding fragment thereof comprises an HCVR having at least 95% identity to SEQ ID NO: 1, and an LCVR having at least 95% identity to SEQ ID NO: 2, and
wherein the antibody or antigen-binding fragment thereof is conjugated to vcMMAE:

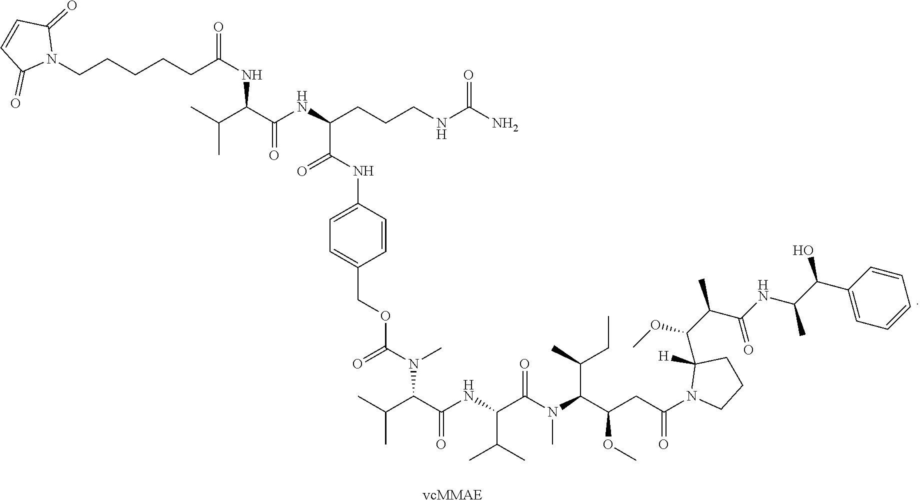 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

23. The method of claim 22, wherein the GCSF is administered prophylactically.

24. The method of any one of claims 19-23, wherein the dose is administered at a concentration of about 2.5 mg/kg of body weight of the subject.

25. The method of any one of claims 19-24, wherein each treatment cycle is administered to the subject Q3W.

26. The method of any one of claims 19-25, wherein a vcMMAE to antibody or antigen-binding fragment thereof ratio is from about 1 to about 8.

27. The method of claim 26, wherein the vcMMAE to antibody or antigen-binding fragment thereof ratio is about 4.

28. The method of any one of claims 19-27, wherein the LIV-1-associated cancer is a breast cancer.

29. The method of claim 28, wherein the breast cancer is a triple negative breast cancer.

30. The method of claim 28, wherein the breast cancer is a metastatic breast cancer.

31. The method of claim 28, wherein the breast cancer is a triple-negative, metastatic breast cancer.

32. The method of claim 28, wherein the breast cancer is a hormone receptor-positive, metastatic breast cancer.

33. A method of treating a subject having or at risk of having a LIV-1-associated cancer, comprising:
administering to the subject a therapeutically effective dose of an antibody or an antigen-binding fragment thereof that specifically binds human LIV-1,
wherein a dose administered is less than about 200 mg of the antibody or antigen-binding fragment thereof per Q3W treatment cycle,
wherein the antibody or antigen-binding fragment thereof comprises an HCVR of SEQ ID NO: 1, and an LCVR of SEQ ID NO: 2, and
wherein the antibody or antigen-binding fragment thereof is conjugated to vcMMAE:

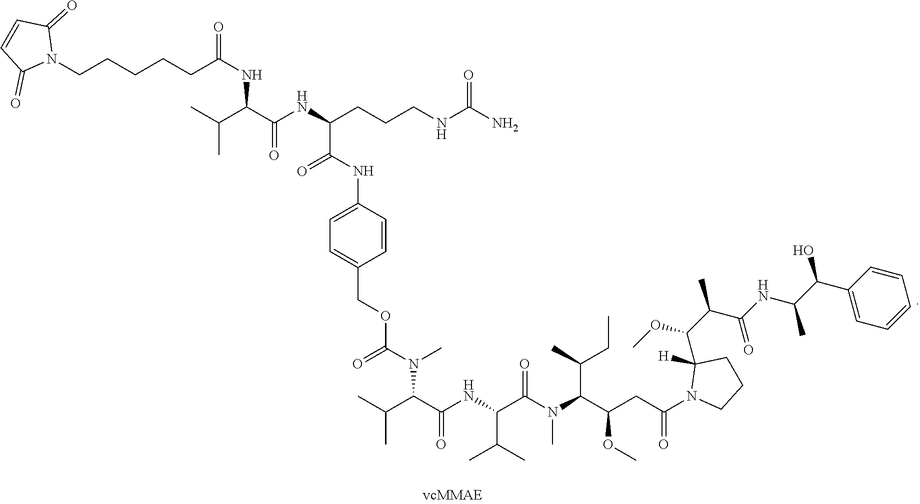 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

34. A method of treating a subject having or at risk of having a LIV-1-associated cancer, comprising:
administering to the subject a therapeutically effective dose of an antibody or an antigen-binding fragment thereof that specifically binds human LIV-1,
wherein a dose administered is less than or equal to about 250 mg of the antibody or antigen-binding fragment thereof per Q3W treatment cycle,
wherein the antibody or antigen-binding fragment thereof comprises an HCVR of SEQ ID NO: 1, and an LCVR of SEQ ID NO: 2,
wherein the antibody or antigen-binding fragment thereof is conjugated to vcMMAE: 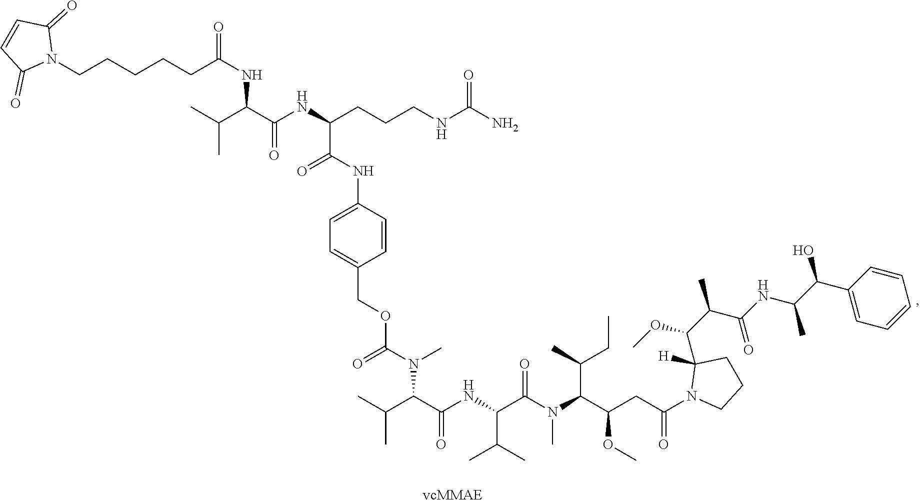 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]
and
wherein if the dose administered is greater than or equal to about 200 mg of the antibody or antigen-binding fragment thereof per treatment cycle, the method further comprises administering granulocyte colony stimulating factor (GCSF) to the subject.

35. The method of claim 34, wherein, if administered, the GCSF is administered prophylactically.

36. A method of treating a subject having or at risk of having a LIV-1-associated cancer, comprising:
administering to the subject granulocyte colony stimulating factor (GCSF),
administering to the subject a therapeutically effective dose of an antibody or an antigen-binding fragment thereof that specifically binds human LIV-1,
wherein a dose administered is greater than or equal to about 200 mg and less than or equal to about 250 mg of the antibody or antigen-binding fragment thereof per Q3W treatment cycle,
wherein the antibody or antigen-binding fragment thereof comprises an HCVR of SEQ ID NO: 1, and an LCVR of SEQ ID NO: 2, and
wherein the antibody or antigen-binding fragment thereof is conjugated to vcMMAE:

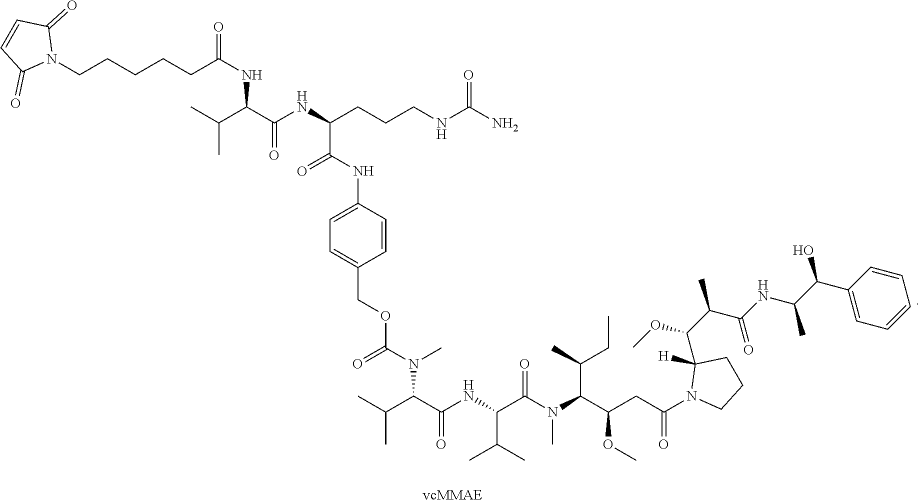 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

37. The method of claim 36, wherein the GCSF is administered prophylactically.

38. The method of any one of claims 33-37, wherein the LIV-1-associated cancer is a breast cancer.

39. The method of claim 38, wherein the breast cancer is a triple negative breast cancer.

40. The method of claim 38, wherein the breast cancer is a metastatic breast cancer.

41. The method of claim 38, wherein the breast cancer is a triple-negative, metastatic breast cancer.

42. The method of claim 38, wherein the breast cancer is a hormone receptor-positive, metastatic breast cancer.

43. The method of any one of claims 33-42, wherein a vcMMAE to antibody or antigen-binding fragment thereof ratio is about 4.

44. The method of any one of claims 33-43, wherein the dose is about 2.5 mg/kg of body weight of the subject.

45. A method of treating a subject having or at risk of having a LIV-1-associated breast cancer, comprising:
administering to the subject a dose of about 2.5 mg/kg of body weight of the subject an antibody or an antigen-binding fragment thereof that specifically binds human LIV-1,
wherein the dose administered is less than about 200 mg of the antibody or antigen-binding fragment thereof per Q3W treatment cycle,
wherein the antibody or antigen-binding fragment thereof comprises an HCVR of SEQ ID NO: 1, and an LCVR of SEQ ID NO: 2, and
wherein the antibody or antigen-binding fragment thereof is conjugated to vcMMAE:

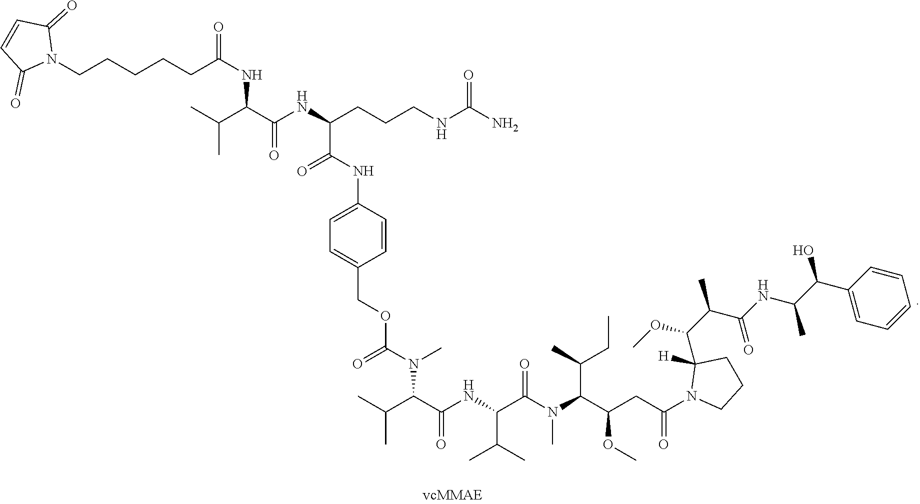 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

46. A method of treating a subject having or at risk of having a LIV-1-associated breast cancer, comprising:
administering to the subject a dose of about 2.5 mg/kg of body weight of the subject an antibody or an antigen-binding fragment thereof that specifically binds human LIV-1,
wherein the dose administered is less than or equal to about 250 mg of the antibody or antigen-binding fragment thereof per Q3W treatment cycle,
wherein the antibody or antigen-binding fragment thereof comprises an HCVR of SEQ ID NO: 1, and an LCVR of SEQ ID NO: 2,
wherein the antibody or antigen-binding fragment thereof is conjugated to vcMMAE: 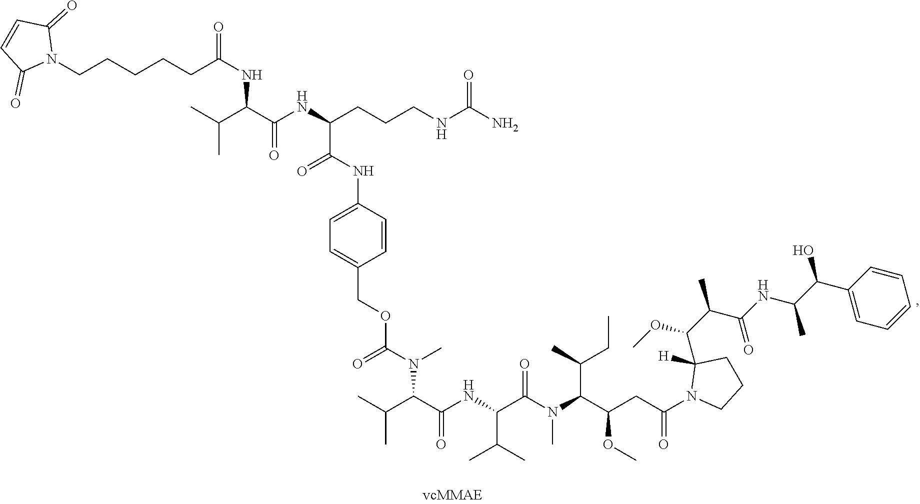 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]
and
wherein if the dose administered is greater than or equal to about 200 mg of the antibody or antigen-binding fragment thereof per treatment cycle, the method further comprises administering granulocyte colony stimulating factor (GCSF) to the subject.

47. The method of claim 46, wherein, if administered, the GCSF is administered prophylactically.

48. A method of treating a subject having or at risk of having a LIV-1-associated breast cancer, comprising:
administering to the subject granulocyte colony stimulating factor (GCSF),
administering to the subject a dose of about 2.5 mg/kg of body weight of the subject an antibody or an antigen-binding fragment thereof that specifically binds human LIV-1,
wherein the dose administered is greater than or equal to about 200 mg and less than or equal to about 250 mg of the antibody or antigen-binding fragment thereof per Q3W treatment cycle,
wherein the antibody or antigen-binding fragment thereof comprises an HCVR of SEQ ID NO: 1, and an LCVR of SEQ ID NO: 2, and
wherein the antibody or antigen-binding fragment thereof is conjugated to vcMMAE:

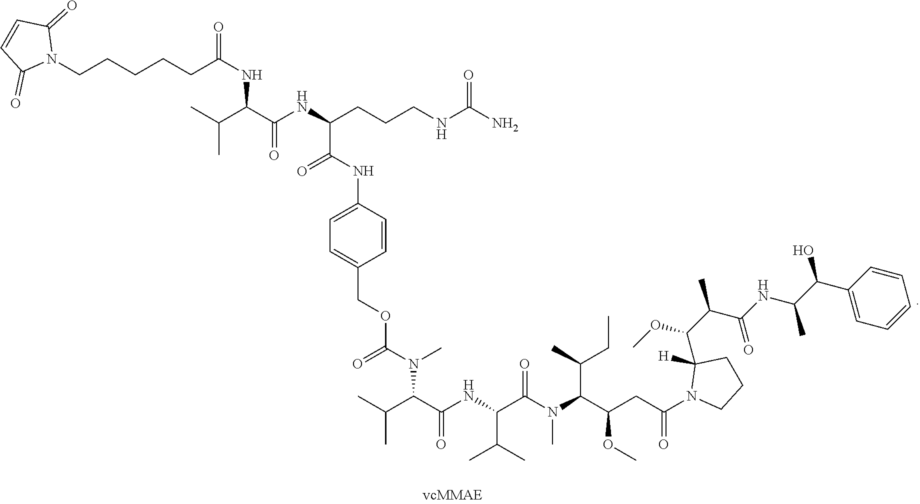 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

49. The method of claim 48, wherein the GCSF is administered prophylactically.

50. The method of any one of claims 45-49, wherein the breast cancer is a triple negative breast cancer.

51. The method of any one of claims 45-50, wherein the breast cancer is a metastatic breast cancer.

52. The method of claim 51, wherein the breast cancer is a triple-negative, metastatic breast cancer.

53. The method of any one of claims 45-50, wherein the breast cancer is a hormone receptor-positive, metastatic breast cancer.

54. The method of any one of claims 45-53, wherein a vcMMAE to antibody or antigen-binding fragment thereof ratio is about 4.

55. The method of any one of claims 1-54, wherein the subject is a human.
